SEQUENCE LISTING (22 sequences)

SEQ ID NO: 1 - DNA, 564 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagattttaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 2 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - DNA, 549 bp, Mus musculus
atgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactga

SEQ ID NO: 4 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 5 - DNA, 39 bp, Artificial Sequence
  Oligonucleotide primer
gcggaattcgccgccaccatgaacaacaggtggatcctc

SEQ ID NO: 6 - DNA, 31 bp, Artificial Sequence
  Oligonucleotide primer
gcggctagctcagttttggaagtttctggta

SEQ ID NO: 7 - DNA, 9 bp, Artificial Sequence
  Consensus Kozak translational initiation signal
gccgccacc

SEQ ID NO: 8 - DNA, 5301 bp, Artificial Sequence
  Plasmid VR4121
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcggatatcgaattcgccgccaccatgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactgagctagcggatcccctagggtcgaccacgtgtgatccagatctacttctggctaataaaagatcagagctctagagatctgtgtgttggttttttgtgtggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 9 - DNA, 39 bp, Artificial Sequence
  Oligonucleotide primer
gcggaattcgccgccaccatgaccaacaagtgtctcctc

SEQ ID NO: 10 - DNA, 29 bp, Artificial Sequence
  Oligonucleotide primer
gcggctagctcagtttcggaggtaacctg

SEQ ID NO: 11 - DNA, 5316 bp, Artificial Sequence
  Plasmid VR6237
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcggatatcgaattcgccgccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgagctagcggatcccctagggtcgaccacgtgtgatccagatctacttctggctaataaaagatcagagctctagagatctgtgtgttggttttttgtgtggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 12 - protein, 13 aa, Homo sapiens
HCLGKWLGHPDKF

SEQ ID NO: 13 - protein, 13 aa, Homo sapiens
HSLGKWLGHPDKF

SEQ ID NO: 14 - protein, 12 aa, Homo sapiens
CFSSSPNEWLQS

SEQ ID NO: 15 - DNA, 2467 bp, Equus caballus
  CDS (697)..(1254)
aagcttcattcctagttttcagttatattgtagatagttgagattgccagataaagcaacaaatgtggctgagaaagctatgtgatgtcttgctgtacatggttggggccctacaaaaaaaatttagatatgcttagttgaacattattcgatgctagatacaaaaaagtaggttgtcttttttaaaatcataaaatgcatatatttattgttttgtgatagtgtaattgggaattaaatctaattcttatgaaaaagaaaattcccatacaaagacccctcaaaaacatctcataatactaaaacaaaaaaataaaaattactttgccaaatgacagaactctgtaatagcagagtcctaaatgatttagcttattttgttccttggtatttaacaatacagtgacactttacaaaacattagaaatcctcagagtttgtatgttttcccctaatatacataaaataaaataggactttaaggatacagagttttagagactacaaataatgtaaatgacataggaaaacagaaagggagaactgaaagtgggaaattcctctgaaatagaaagagtggaggaccatcccgtataaatagcccacactcacggaggaaggacatttaagctcaagccgttgccacctccacttgggctcctagggagtaaaggcaacactgttcctgtcttcatcatgacctacaggtggatcctcccaatggccctcctgctgtgtttctccaccacggctctttctgtgaactatgacttgcttcggtcccaactaagaagcagcaattcagcatgtctgatgctcctgcggcagttgaatggagcccctcaacgttgccccgaggacacaatgaacttccaggtccctgaggagattgagcaagcacagcagttccagaaggaggatgctgcattggtcatctatgagatgctccagcacacctggcgtattttcagaagaaatttcgctagcactggctggaatgagaccatcgttaagaacctccttgtggaagtccatctgcagatggaccgtctggagacaaacctggaggaaataatggaggaggaaagctccacctggggaaacacaaccattctgcgcctgaagaaatactacggaaggatctcgcagtacctgaaggccaagaagtacagccactgtgcctggacagtggtccaagcggaaatgctcaggaacttggccttccttaacggactcacagattacctccaaaactgaggatctcccagcctgcacctcggagaagggacaatgctgacagtgactgcaggtgtcttcccagcagaggctcttgacgtgactgacagctaaggcactgcattggaaaggacagttacagactttacattttttactaacttatgaattaacttatttttctatttatttcaacatttaccttggaaaataaattttttatgaaacaaaattcaacacggctgttttaatttcaacttgatttatagaatcacccagattaaaaactgcaaaccacctgtaaaatgttctttgtaaaatgtgcctgcaaactagtatagtttctggcccctgccttcaaggaatttaaaatccaaggaagccatgcggaatatacaagataagaggtgagaaggggacctcaaccgtacaggagaagaaatgtggcttgagccccatataaacggaattaaaatgggagagacaggcagaggctctggactcagaggacggggctgctgcttctgccctgtgtcccgctctctggccccacagttagaatctgatggctctcagggtgcccagaggaatatgtcagctcttgcgtttgcctggagctcatccctactatctgcgagatgctctgcctccccccacccctcaaccccacaggattgtaaaatatttctgtgccctgcaagcctaagcgggagaagtcccaggcacttctgggacactgtaagtggcagtccctttatggtactcttcttgggacaaccgagctgtacaggtgtctaaggggagccagcgtctctgtctccttccagggcacagacacaagaggaagaaagaactctgtttcatacccctgccatcggcctggttttgctccctatttttccagagaagcaagtctgctccggcttttcctgctgctctgcgtctccaggccacactctccccaaagccaaggccaaggcaggtgcagcagattaggtccagtctcaggccagtgagaaaccgggaagcatgggagacaaggaaattcaggtgggatagagagggcactaacgttcccagggcttacactgggaaattggagatttcctaggagctctttgggcaccggccagcatagctgcttttctgtctgtgctgaacccttgggaacgtgcattattatgcctgttcttgccatgagcaggggatccgtcgacctgcagccaagctt

SEQ ID NO: 16 - protein, 186 aa, Equus caballus
MTYRWILPMALLLCFSTTALSVNYDLLRSQLRSSNSACLMLLRQLNGAPQRCPEDTMNFQVPEEIEQAQQFQKEDAALVIYEMLQHTWRIFRRNFASTGWNETIVKNLLVEVHLQMDRLETNLEEIMEEESSTWGNTTILRLKKYYGRISQYLKAKKYSHCAWTVVQAEMLRNLAFLNGLTDYLQN

SEQ ID NO: 17 - DNA, 2103 bp, Sus scrofa
  CDS (1266)..(1823)
ggatccatgagagtgaggattacaagctgcagcagctaatgctacaatggtacacagcgtgggtttagtaaatttggtttgaaaaataacacaaggttttctgaaaactgaaaatcattacttaaaatattataattattagaacttctctttaaagtgtactatatgtctcctgtctccttataggcaggacataaaatcataatttgtaatttttacttactcctctcatcaaaatctgttactacactctgagtaacaggactcagaaatattgagctaagatccttatctcaacagtaacatcacctattaatgcttgataatttttctgtccattttccttccagacctccttctaataaatggagtatatttgcatgtgaatggaaaatttaaaaatttagattgttagaagtatgtgcaaaataaaaatacattggccctaaataatggtcacaaaggccccctaaaagggaatcatgaacttttatgcatggcccatgttacattcattttacctccttagcgatattacttaatagacattaagttatattctgatagttgagactgccagaaaaaaagtgataaattaagtcgcataaaatcatgtgatgtcttgctgcttatggttagggccctacaaaaaaaattcagatcaacttactttaacagtattctattttagatagaaagttggtatcttgtgaagtcatagacttcatgtatttattgttttgtaatagtgtaactgggaattaaatataattcttatgaaaaagaaaactctcttaaaagcaacccccccaaaatctcgtaaatatgaaaaaagaaaaagagaaatctttgccaaatgaaagaaccctgaaacaatcaagctcaaaatgacttggcttatggtggttttttttgcacttacagcatattttagtgtttattagttcacaaatactttccaaaacatccaaaatccccagttcactaaaactttaccaaatgtaagaattcctccaaatgtgtataacttttgttccctaatatatgtgaaataaaatagtgttggatgaatgctaacaaactcaaatgacataggaaaactgaaagggagaactgaaagtgggaaattcctctgaactagaaagagtggagggccttccagtataaatagcctatggagaaagaacattcacactgcacactcctgaagacttcacttcagcacttgagtagtggagccagtaaccgtgtttccgttttcatcaatggctaacaagtgcatcctccaaatcgctctcctgatgtgtttctccaccacagctctttccatgagctatgatgtgcttcgataccaacaaaggagcagcaatttggcatgtcagaagctcctgggacagttgcctgggactcctcaatattgcctcgaagataggatgaactttgaggtccctgaggagattatgcaaccaccacaattccagaaggaagatgcagtattgattatccacgagatgctccagcagatcttcggcattctcagaagaaatttctctagcactggctggaatgaaaccgtcattaagactatccttgtggaacttgatgggcagatggatgacctggagacaatcctggaggaaatcatggaggaggaaaatttccccaggggagacatgaccattcttcacctgaagaaatattacttgagcattctgcagtacctgaagtccaaggagtacagaagctgtgcctggacagtcgtccaagtggaaatcctcaggaacttttctttccttaacagacttacagattacctccggaactgaacatctcccccctgtggctctgggaattgaccatgttggcaatgatgtcaggctcttcaagcaggggaagctctttcagtgactgacagacaatgcactgaatttgaatggactgttaaagacttttagcttttttaataataatttatgcattaaattatgtatttaattttttaccttggtggattttctgtgtgaatcggcgggttacgaacctgacttgtatccatgaggatgtgagtttgatctgaggcctggatcagtgggttaaggatcc

SEQ ID NO: 18 - protein, 186 aa, Sus scrofa
MANKCILQIALLMCFSTTALSMSYDVLRYQQRSSNLACQKLLGQLPGTPQYCLEDRMNFEVPEEIMQPPQFQKEDAVLIIHEMLQQIFGILRRNFSSTGWNETVIKTILVELDGQMDDLETILEEIMEEENFPRGDMTILHLKKYYLSILQYLKSKEYRSCAWTVVQVEILRNFSFLNRLTDYLRN

SEQ ID NO: 19 - DNA, 652 bp, Felis catus
  CDS (86)..(646)
tggaagaagggcattcacactgcaaactctcgaagtctttgcttcagcacctagacagtagcaggcaagacttcctattttcatcatgaccggcaggtgcatcctccaaatcgctctcttggtgtgtttcttcaccaccgcgcattccgtgagctacaagttgcttggattccaactaagaagcagcagtttggagtgtcaggagctcctggtgaacttgaacagaacctctaaatattgcctcaaggacaggatgaacttcgaggtccctgaggagattaaaaaatcacagcggttccagaaggaggaagccatattggtcatcaacgagatgttccagaagatctttaatattttcagtagaagcacctctagcacgggatggaatgagaccactgttgagaacctccttgcgacactccactggcagaaggaacacctggaaacgatcctggaggaaatcatggaggaggaaaacttcacctgggacaatacgacccttctgaacctgaagaaatactacttaaggattgtgcggtacctgaaggccaaggagtacagcgtctgtgcctggacagtagtccacgcagaaatcctcagaaactttttcttccttgagagacttacagattacctccaaaactgaggacct

SEQ ID NO: 20 - protein, 186 aa, Felis catus
MTGRCILQIALLVCFFTTAHSVSYKLLGFQLRSSSLECQELLVNLNRTSKYCLKDRMNFEVPEEIKKSQRFQKEEAILVINEMFQKIFNIFSRSTSSTGWNETTVENLLATLHWQKEHLETILEEIMEEENFTWDNTTLLNLKKYYLRIVRYLKAKEYSVCAWTVVHAEILRNFFFLERLTDYLQN

SEQ ID NO: 21 - DNA, 959 bp, Rattus norvegicus
  CDS (306)..(860)
tggtaattaatgaaactgcataaagtttttataaatctctacagtttgcatacattttaatccagtgaatagtatataaaatagccaggagcttaaataaaatgaatattagaagctgttagaataagagaaaaatgacggaggaaaactgaaagggagaactgaaagtgggaaattcctctgaggcagaaaggaccatcccttataaatagcacagaccatgaaggaagatcattctcactgcagcctttggtagcctttgcctcatcgtgcaggtagcagccaacaccagcccagcttccatcatggccaacaggtggaccctccacattgcgttcctgctgtgcttctccaccactgccctctccatcgactacaagcagctccagttccgacaaagcactagcattcggacatgtcagaagctcctgaggcagctgaatggaaggctcaacctcagctacaggacggacttcaagatccctatggaggtgatgcacccgtcacagatggagaagagttacactgcctttgccattcaagtgatgctccagaatgtctttcttgtcttcagaagcaatttctccagcactgggtggaatgagactattgttgaaagtctcttggatgaactacatcagcagacagagcttctggagataatactaaaggaaaagcaagaggaaagattgacttgggtgacatccacgactactttaggcttgaagagctattactggagggtacaaaggtaccttaaagacaagaagtacaacagctatgcctggatggtggtccgagcagaagtcttcaggaacttttccattattctaagacttaatagaaacttccagaactgaagacctgtcagccaatgcctccaagagcaggtgatggttgcaggcaatcttaaaacattagagtctgactctgtgactggtagtgaatctactgcattt

SEQ ID NO: 22 - protein, 184 aa, Rattus norvegicus
MANRWTLHIAFLLCFSTTALSIDYKQLQFRQSTSIRTCQKLLRQLNGRLNLSYRTDFKIPMEVMHPSQMEKSYTAFAIQVMLQNVFLVFRSNFSSTGWNETIVESLLDELHQQTELLEIILKEKQEERLTWVTSTTTLGLKSYYWRVQRYLKDKKYNSYAWMVVRAEVFRNFSIILRLNRNFQN

(19) United States
(12) Patent Application Publication
(10) Pub. No.: US 2003/0091544 A1
(43) Pub. Date: May 15, 2003

(54) Interferon-Beta polynucleotide therapy for autoimmune and inflammatory diseases

(75) Inventors: Suezanne E. Parker, San Diego, CA (US); Holly M. Horton, San Diego, CA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Vical Incorporated

(21) Appl. No.: 10/096,373

(22) Filed: Mar. 13, 2002

Related U.S. Application Data
(60) Provisional application No. 60/275,044, filed on Mar. 13, 2001.

(51) Int. Cl. A61K 48/00

(52) U.S. Cl. 424/93.21; 514/44

(57) ABSTRACT

The present invention relates to methods of treating an autoimmune disease or an inflammatory condition, and in particular multiple sclerosis, in a mammal comprising administering a therapeutically effective amount of a non-infectious, non-integrating polynucleotide construct encoding a β interferon or an active fragment or variant thereof, wherein said construct is not associated with transfection-facilitating viral particles, liposomal formulations, or charged lipids.

### CROSS-REFERENCE TO RELATED APPLICATIONS

The present application claims benefit of the filing date of U.S. Provisional Application No. 60/275,044, filed Mar. 13, 2001, which is incorporated herein by reference in its entirety.

### BACKGROUND OF THE INVENTION

1. Field of the Invention

The present invention relates to treatment of autoimmune diseases and other conditions related to inflammation in mammals. Generally, the present invention provides methods of treating or preventing autoimmune diseases and other conditions related to inflammation, caused, for example, by inflammatory demyelinating diseases, in a mammal by administering a polynucleotide or polynucleotide construct comprising a polynucleotide encoding interferon-beta (IFNβ), or an active fragment or variant thereof. In certain embodiments, the methods involve delivery of a polynucleotide or polynucleotide construct encoding IFNβ or an active fragment or variant thereof, where the polynucleotide or polynucleotide construct is not associated with transfection-facilitating viral particles, liposomal formulations, or charged lipids. Alternatively, the polynucleotide or polynucleotide construct encoding IFNβ or an active fragment or variant thereof may be delivered as part of a composition comprising, for example, certain transfection-facilitating auxiliary agents such as poloxamers.

2. Background Art

Multiple sclerosis (MS) involves demyelination of neurons of the central nervous system (CNS) and affects 250,000 individuals in the United States (Bansil, S., et al., Ann. Neurol. 37:S87-S 101 (1995); Steinman, L., Cell 85:299-302 (1996); Noseworthy, J. N., et al., NEJM. 343:938-952 (2000)). With destruction of the protective myelin sheath, nerve impulses are disrupted leading to a variety of neurological symptoms, such as lack of coordination, vision disturbances, loss of sensation, weakness, gait abnormality and, in later stages, various degrees of paralysis. The disease is more common in women (1.6:1 female:male) and is more common among populations residing in the Northern hemisphere. A genetic link has not been conclusively identified although a preference for certain HLA genes has been found and there is a higher shared incidence among monozygotic twins, compared to dizygotic twins or siblings.

The majority of patients have relapsing-remitting MS in which neurological dysfunction is followed by periods of recovery. As the disease progresses, patients may eventually develop secondary progressive MS in which the disease worsens and periods of recovery are absent. Demyelinating lesions or plaques can be followed in MS patients using magnetic resonance imaging (MRI). Plaques or areas of demyelination are often observed during periods of disease exacerbation while reductions in plaques, during periods of remyelination, may be associated with symptom-free periods. A minority of patients initially develop primary progressive MS in which the disease progresses steadily with no periods of recovery.

Studies using a mouse model of MS, experimental allergic encephalomyelitis (EAE) (Alvord, E. C., et al., Prog. Clin. Biol. Res. 146:1-8 (1984); Swanborg, R. H., Clin, Immunol. Immunopathol. 77:4-13 (1995); Martin, R, and McFarland, H. F., Crit. Rev. Clin. Lab. Sciences 32:121-182 (1995)), have been useful in characterizing the immune response in a disease similar to MS. EAE can be induced in several strains of mice by subcutaneous (s.c.) injection of myelin proteins such as myelin basic protein (MBP) or proteolipid proteins (PLP) in the presence of Freund's adjuvant. Adoptive transfer studies in the EAE model demonstrated that CD4+ T cells from mice immunized with MBP or PLP could transfer disease to naive mice suggesting that EAE is a T cell-mediated disease. Specific encephalitogenic epitopes in MBP and PLP have been identified and most of the responses are restricted by 1A-molecules, the murine counterpart to human HLA-DQ.

In EAE, encephalitogenic T cells enter the CNS and initiate a delayed type hypersensitivity (DTH) response characterized by secretion of T helper type I (TH1) cytokines such as TNFα and IFNγ. TNFα can be cytotoxic to oligodendrocytes and myelin sheaths and damage endothelial cells resulting in leakage of the blood brain barrier (Selmaj, K., and Raine, Ann. Neurol. 23:339-346 (1988); Zajicek, J. P., et al., Brain 115:1611-1631 (1992)). IFNγ may upregulate MHC Class I and II on astrocytes, microglia and endothelial cells and can activate macrophages, NK cells and stimulate the differentiation of cytotoxic T cell (CTL) precursors to CTL (Welsh, J., et al., J. Neuroimmunol. 48:91-98 (1993); Farrar, M. A., and Schreiber, R. D., Annu. Rev. Immunol. 11:571-612 (1993)).

Although MS is generally considered to be an autoimmune disease, the triggering and target antigens have not yet been conclusively identified. However, T cells reactive to MBP, PLP and myelin-oligodendrocyte glycoprotein (MOG) were found at higher levels in MS patients compared to normal patients (Olsson T., et al., J Clin. Investig. 86:981-985 (1990); Sun, J. B., et al., Eur. J. Immunol. 21:1461-1468 (1991a); Sun, J. B., et al., J. Immunol. 146:1490-1495 (1991b)). CNS lesions in MS appear to be infiltrated with CD4+ and CD8+ T cells and macrophages and MS patients often have increased serum levels of IL-2, IL-12 and TNFα (Gallo, P., et al., J. Neurol. Sci. 92:9-15 (1989); Sharief, M. K., et al., J. Neuroimmunol. 43:15-22 (1993); Tsukada, N., et al., J. Neurosci. 102:230-234 (1991)). Treatment of MS patients with the TH-1 cytokine interferon γ (IFNγ) was found to exacerbate the disease (Panitch, H. S., et al., Neurology 37:1097 (1987)).

Therapies for MS

Since the triggering antigen has not been identified for MS, current therapies are directed at reducing the symptoms of the disease. Corticosteroids are commonly prescribed during acute attacks due to their anti-inflammatory and immunosuppressive properties but they have little effect on progressive disease. Another therapy is copolymer-1, an amino acid copolymer, which may compete with MBP for MHC Class II binding sites. Other immunomodulating therapies include cyclophosphamide, cyclosporine and mitoxantrone, although the efficacy of these therapies is not well-established (Bansil, S., et al., Ann. Neurol. 37:S87-S101 (1995)).

Recombinant IFNβ protein was approved for human use after a series of landmark clinical studies demonstrating the efficacy of this cytokine (The IFNβmultiple sclerosis study group, 1993 and 1995). Two forms of IFNβ have been approved. Betaseron (IFNβ-1b, Berlex) has a one amino acid change from native IFNβ, while Avonex (IFNβ-1a, Biogen) lacks the amino acid change and is the same as natural IFNβ. IFNβ-1b administered s.c. to MS patients every other day reduced the frequency and severity of relapses and decreased the number and severity of lesions on MRI (IFNβ MS study group, Neurology 43:665-661 (1993); IFNβ MS study group, Neurology 45:1277-1285 (1995)). In another study, IFNβ-1a administered i.m. weekly resulted in a significant slowing in accumulation of disability and led to significantly fewer exacerbations and smaller brain lesions (Jacobs, L. D., et al., Annals of Neurology 39:285-294 (1996)). It was recently demonstrated that IFNβ-1b can delay progression of secondary progressive MS (Kappos, L., Lancet 352:1491-1497 (1998)). More recently, in a trial of high-risk patients with early-stage disease, IFNβ-1a delayed MS or prevented it from developing (Jacobs, L. D., et al., NEJM. 343:898-904 (2000)). In this three year study of 383 patients, the patients that received weekly i.m. injections of IFNβ-1a protein, rather than a placebo, were half as likely to develop further disease and had smaller lesions in the CNS. The results of this study suggested for the first time that early treatment of MS patients with IFNβ may lessen disease progression.

The mechanism by which IFNβ reduces the severity of MS is not certain. However, IFNβ antagonizes the actions of IFNγ, such as the IFNγ-dependent upregulation of MHC Class II expression (Barna, B. P., et al., J Neuroimmunol. 23:45-53 (1995)). IFNβ also inhibits the production of IFNγ and TNFα by T cells or peripheral blood mononuclear cells (PBMC) (Noronha, A., et al., J. Neuroimmunol. 46:145-154 (1993); Rudick, R. A., et al., Neurology 43:2080-2087 (1993)). Recently, IFNβ was found to inhibit IL-12 production by PBMC and by MBP-specific T cell lines and to increase the secretion of the TH-2-type cytokine IL-10 by T cells and monocytes (Wang, X., et al., J. Immunol. 165:548-557 (2000); Rep, M. H. G., et al., J. Neuroimmunol. 67:111-118 (1996); Rudick, R. A., et al., Ann. Neurol. 40:618-627 (1996)). Thus, IFNβ may be involved in a switch from TH-1 type responses to TH-2 type responses in the CNS which may be therapeutic for MS.

Gene Therapy of MS

In preclinical studies of MS, plasmid DNA (pDNA) has been used to deliver immunomodulatory cytokines. In these studies, pDNA was delivered by either intramuscular (i.m.) injection of naked pDNA or intracranial (i.c.) injection of pDNA complexed with lipid for therapy of EAE. A single i.e. injection of 100 ug of pDNA encoding either IFNβ, IL-4, TGF-β or a TNF receptor (TNFR)/Ig fusion complexed with lipid, 12 days after disease induction, was found to significantly reduce the clinical score of mice with EAE (Triantaphyllopoulos, K. A., et al., Gene Ther. 5:253-263 (1998); Croxford, J. L., et al., J. Immunol. 160:5181-5187 (1998)). Delivery of the latter constructs by i.m. injection of naked pDNA, however, was not effective in treating the disease (Croxford, J. L., et al., J. Immunol. 160:5181-5187 (1998)). In a later study, i.m. delivery of a different pDNA vector encoding TGF-β or IL-4 on days −2 and +5, relative to disease induction, resulted in a significant reduction in symptoms (Piccirillo, C. A., et al., J. Immunol. 161:3950-3956 (1998)).

Guillain-Barre Syndrome (GBS)

Guillain-Barre Syndrome (GBS) is manifested as autoimmune inflammation of the peripheral nervous system in human patients. Studies using a mouse model of GBS have evaluated the impact of cytokine therapy on disease progression. Specifically, recombinant IFNβ (300,000 U) of was i.m. injected at the onset of experimental autoimmune neuritis (EAN) disease development, resulting in disease amelioration (Zou, L. P., et al., J. Neurosci. Res. 56(2): 123-30 (1999)). Since some patients have experienced flu-like symptoms after injection of IFNβ protein (Lublin, F. D., et al., Neurology 46:12-18 (1996)), treatment with IFNβ pDNA may result in fewer side-effects. The side effects of IFNβ protein therapy may be related to the high serum levels occurring in the first 8-48 hrs after injection of the protein (Chiang, J., et al., Pharmaceutical Research 10:567-572 (1993); Alam, J., et al., Pharmaceutical Research 14:546-549 (1997)). Lower, more stable serum levels may be achieved upon injection of IFNβ pDNA.

Rheumatoid Arthritis

Rheumatoid arthritis is an inflammatory condition that may also benefit from IFNβ treatment. In an animal model of rheumatoid arthritis called Collagen-Type II Induced Arthritis (CIA), in rhesus monkeys (Macaca mulatta) were injected with 10×10⁶ units (MIU)/kg body weight of CHO cell-derived human recombinant IFNβ-1a (Rebif®; Ares-Serono, Geneva, Swizerland) s.c., at at the onset of CIA disease development daily for 1 week, resulting in rapid clinical improvement during therapy and a decrease in serum C-reactive protein (CRP) levels (Tak, P., et al., Rheumatology 38:362-369 (1999)). However, the discontinuation of therapy resulted in the subsequent increase in CRP levels and relapse of CIA disease. The use of IFNβ pDNA instead of recombinant IFNβ would permit a longer-term delivery of IFNβ, therefore permitting a longer course of therapy without requiring daily injections.

A similar treatment protocol was carried out in patients presenting with rheumatoid arthritis, using purified native human, natural fibroblast IFNβ (Frone®; Arcs-Serono), which was self-administered by the patients s.c. three times weekly for 12 weeks at the following dosages: 6 MIU, 12 MIU, and 18 MIU (Tak, P., et al., Rheumatology 38:362-369 (1999)). Patients developed flu-like symptoms however they exhibited statistically significant gradual improvement in tender joint count, swollen joint count, patient's assessment of pain, patient's global assessment and physician's global assessment. The duration of morning stiffness and serum levels of serum C-reactive protein (CRP) were generally lower after IFNβ treatment, however these results were not statically significant. Three months after initiation of treatment, 4 patients fulfilled the American College of Rheumatology (ACR) criteria for 20% improvement however none of the patients fulfilled the ACR criteria for 50% improvement. The use of IFNβ pDNA instead of recombinant IFNβ may result in fewer side-effects and may allow for the localized delivery of greater dosages of IFNβ, and may increase increase improvement % based on ACR criteria.

### BRIEF SUMMARY OF THE INVENTION

The present invention is broadly directed to treatment of autoimmune diseases and other conditions related to inflammation by administering in vivo, into a tissue of a mammal suffering from autoimmune diseases and other conditions related to inflammation, a IFNβ-encoding polynucleotide, polynucleotide construct or an active fragment or variant thereof, or composition comprising an IFNβ polynucleotide or polynucleotide construct or an active fragment or variant thereof. The polynucleotide or polynucleotide construct is incorporated into the cells of the mammal in vivo, and a therapeutically effective amount of an IFNβ or active fragment or variant thereof is produced in vivo.

The present invention provides a method of treating, preventing, or reducing the symptoms of autoimmune diseases and other conditions related to inflammation in a mammal comprising administering to said mammal a polynucleotide or polynucleotide construct, in certain embodiments, a non-infectious, non-integrating polynucleotide construct comprising a polynucleotide selected from the group consisting of (a) a polynucleotide that hybridizes under stringent conditions to the nucleotide sequence of SEQ ID NO:1 or the complement thereof, wherein the polynucleotide sequence encodes a polypeptide that has anti-viral and/or anti-proliferative activity; (b) a non-infectious, non-integrating polynucleotide construct that encodes a polypeptide comprising an amino acid sequence which, except for at least one but not more than 20 amino acid substitutions, deletions, or insertions, is identical to amino acids −21 to 166, 1 to 166, or 2 to 166 in SEQ ID NO:2, wherein the polypeptide has anti-viral and/or anti-proliferative activity; and (c) a polynucleotide encoding encoding IFNβ or an active fragment or variant thereof, wherein said construct is free from transfection-facilitating viral particles, liposomal formulations, or charged lipids.

In certain embodiments, the present invention provides a method of treating, preventing, or reducing the symptoms of autoimmune diseases and other conditions related to inflammation in a mammal, for example, treating, preventing, or reducing the symptoms an inflammatory demyelinating disease in a mammal comprising administering in vivo into a tissue of a mammal, preferably a mammal in need of such treatment, a composition comprising a polynucleotide or polynucleotide construct encoding interferon-beta (IFNβ), or an active fragment or variant thereof, together with a pharmaceutically acceptable carrier, where the IFNβ-encoding polynucleotide or polynucleotide construct is either a DNA plasmid encoding said IFNβ or active fragment or variant thereof through operable association with a promoter or a messenger RNA, and where the IFNβ-encoding polynucleotide or polynucleotide construct is free from association with liposomal formulations and charged lipids. In this embodiment, the polynucleotide or polynucleotide construct is incorporated into the cells of said mammal, and a therapeutically effective amount of IFNβ, or active fragment or variant thereof is expressed.

In additional embodiments, the present invention provides a method of treating, preventing, or reducing the symptoms of autoimmune diseases and other conditions related to inflammation in a mammal, for example, treating, preventing, or reducing the symptoms an inflammatory demyelinating disease in a mammal comprising administering in vivo into a tissue of a mammal, preferably a mammal in need of such treatment, a composition comprising a polynucleotide or polynucleotide construct encoding interferon-beta (IFNβ), or an active fragment or variant thereof, together with a pharmaceutically acceptable carrier, where the IFNβ-encoding polynucleotide or polynucleotide construct is either a DNA plasmid encoding said IFNβ or active fragment or variant thereof through operable association with a promoter or a messenger RNA, and where the tissue of administration is either muscle, skin, or blood. In this embodiment, the polynucleotide or polynucleotide construct is incorporated into the cells of said mammal, and a therapeutically effective amount of IFNβ, or active fragment or variant thereof is expressed.

Inflammatory demyelinating diseases to be treated by the methods of the present invention include, but are not limited to multiple sclerosis, Guillain-Barre Syndrome, experimental autoimmune encephalomyclitis and experimental autoimmune neuritis.

According to the present invention, “polynucleotides encoding interferon-beta (IFNβ), or active fragments or variants thereof” include, but are not limited to: (a) a polynucleotide comprising a nucleic acid that hybridizes under stringent conditions to the complement of SEQ ID NO:1, wherein the polynucleotide sequence encodes a polypeptide that has anti-viral or anti-proliferative activity; (b) a polynucleotide comprising a nucleic acid that encodes a polypeptide which, except for at least one but not more than 20 individual amino acid substitutions, deletions, or insertions, is identical to amino acids 1 to 166 in SEQ ID NO:2, wherein the polypeptide has anti-viral or anti-proliferative activity; and (c) a polynucleotide comprising a nucleic acid that encodes a polypeptide at least 80, 85%, 90%, 91%, 92%, 93%, 94%, 95%, 96%, 97%, 98%, or 99% identical to amino acids 1 to 166 in SEQ ID NO:2, wherein the polypeptide has anti-viral or anti-proliferative activity.

In addition, the present invention provides a method of treating, preventing, or reducing the symptoms of multiple sclerosis in a mammal, comprising administering to said mammal an IFNβ-encoding polynucleotide or polynucleotide construct, wherein said construct is not associated with transfection-facilitating viral particles, liposomal formulations, or charged lipids.

Compared to injection of recombinant cytokine polypeptides, the methods described herein have several important advantages. The present invention shows that in vivo transfection of cells with IFNβ-encoding polynucleotide or polynucleotide construct results in serum levels of IFNβ that have therapeutic effects, and yet are lower than the maximal serum levels typically required when IFNβ polypeptides are injected. Further, injecting frequent high doses of IFNβ polypeptide can produce debilitating side effects. The methods of the present invention provide IFNβ therapy requiring less frequent injections of IFNβ-encoding nucleic acids where in vivo transfection of cells with IFNβ-encoding polynucleotide or polynucleotide constructs results in therapeutic effects. The injection of polynucleotide or polynucleotide constructs encoding IFNβ produces sustained, moderate levels of biologically active IFNβ that have beneficial effects, while minimizing adverse side effects.

### BRIEF DESCRIPTION OF THE DRAWINGS/FIGS

FIG. 1. IFNβ pDNA therapy of EAE. For induction of disease, SJL/J mice were injected s.c. with MBP and Mycobacterium tuberculosis (M Tb.) on days 0 and 7. On days −2 and +5, relative to the first MBP injection, mice were injected i.m. with 100 μg of mIFNβ pDNA, control pDNA or with saline (n=15 mice per group). A significant reduction in neurological score for both the primary attack and during the relapse was found for the mice treated with mIFNβ pDNA (p<0.05).

FIG. 2. Plasmid map of VR4121 (SEQ ID NO:6). The cytomegalovirus immediate early gene promoter enhancer and 5′ untranslated sequences (5′ UTR+intron A) drive the expression of the mouse IFNβ coding sequence. The transcriptional terminator region includes a polyadenylation and termination signal derived from the rabbit β-globin gene.

FIG. 3. Plasmid map of VR6237 (SEQ ID NO:9). The cytomegalovirus immediate early gene promoter enhancer and 5′ untranslated sequences (5′ UTR+intron A) drive the expression of the human IFNβ coding sequence. The transcriptional terminator region includes a polyadenylation and termination signal derived from the rabbit P-globin gene.

FIG. 4. IFNβ pDNA treatment of primary attack. For induction of disease, SJL/J mice were injected s.c. with PLP peptide (SEQ ID NO:12) and Mycobacterium tuberculosis (M Tb.) on day 0. On days 2 and 5, relative to the first PLP injection, mice were injected i.m. with 100 fig of mIFNβ pDNA, or control pDNA (n=14 mice per group). A significant reduction in neurological score for the primary attack was found for the mice treated with mIFNβ pDNA (p<0.05).

FIG. 5. IFNβ pDNA therapy of relapse. For induction of disease, SJL/J mice were injected s.c. with PLP peptide (SEQ ID NO: 13) and Mycobacterium tuberculosis (M Tb.) on day 0. On days 18, 21, 25, 28, and 32 relative to the first PLP injection, mice were injected i.m. with 100 μg of mIFNβ pDNA, or control pDNA (n=9 mice per group). A significant reduction in neurological score for the relapse was found for the mice treated with mIFNβ pDNA (p<0.05).

### DETAILED DESCRIPTION OF THE INVENTION

The present invention is broadly directed to treating, preventing or reducing the symptoms of an autoimmune disease or an inflammatory condition in a mammal by administering in vivo, into a tissue of a mammal, at least one polynucleotide or polynucleotide construct comprising at least one polynucleotide encoding IFNβ, or at least one active fragment or variant thereof. In certain embodiments, the polynucleotide or polynucleotide construct encoding IFNβ is delivered as part of a pharmaceutical composition, in which the polynucleotide is dissolved in a salt solution, e.g. 150 mM sodium phosphate. Certain polynucleotides or polynucleotide constructs may include auxiliary agents including, but not limited to, poloxamers, DMSO, IGEPAL® CA 630, NONIDET NP-40®, Nonidet P40, Triton X-100™, Triton X-114™, sodium dodecyl sulfate, Tween-20®, Tween-80®, stachyose, EDTA, Thesit®, combinations thereof, and reaction, association, or dissociation products thereof. In certain embodiments, the polynucleotide or polynucleotide construct, or a pharmaceutical composition comprising the polynucleotide or polynucleotide construct, is not associated with transfection-facilitating viral particles, liposomal formulations, or charged lipids. In a preferred embodiment, the polynucleotide or polynucleotide construct is incorporated into the cells of the mammal in vivo, and a therapeutically effective amount of IFNβ is produced in vivo, to treat or prevent autoimmune diseases and other conditions related to inflammation

Experimental allergic encephalomyelitis (EAE), a murine model of an autoimmune inflammatory demyelinating disease of the CNS, has been accepted as providing an excellent model to assess interventions to alter the course of human MS. EAE develops in animals injected with spinal cord proteins, and can also be induced by passive transfer of T-cell clones made reactive for certain myelin antigens (e.g. myelin basic protein). Parenteral (IV) natural rat fibroblast interferon (10⁵ units) can suppress partially acute EAE in male Lewis rats (Abreu et al., Immunol. Commun., 11:1-7 (1982)); and inhibit passive hyperacute localized EAE when administered on the same day as immunogen inoculation (Abreu et al., Int. Arch. Allergy Appl. Immunol., 72:30-33 (1983)). Other parenterally administered cytokines, such as TGF-β can decrease clinical disease and inflammation in brain and spinal cord in EAE (Johns et al., J. Immunol. 147:1792-1796 (1991)). In the mouse model of EAE, native IFNβ protein administered on the day of EAE onset and every other day thereafter (5000 or 10,000 IU IFNβ) resulted in decreased clinical scores and a delay in progression of the disease (Yu, et al., J. Neuroimmunol., 64:91-100 (1996)). The present inventors have shown that the administration of naked IFNβ DNA is effective for treatment of EAE.

The present invention provides a method of treating or preventing an autoimmune disease or an inflammatory condition in a mammal comprising administering to said mammal a polynucleotide or polynucleotide construct, in certain embodiments, a non-infectious, non-integrating polynucleotide or polynucleotide construct, encoding an IFNβ or an active fragment or variant thereof. In some embodiments, the IFNβ-encoding polynucleotide or polynucleotide construct is not associated with transfection-facilitating viral particles, liposomal formulations, or charged lipids. In other embodiments, the IFNβ-encoding polynucleotide or polynucleotide construct is administered as part of a pharmaceutical composition when the polynucleotide or polynucleotide construct is dissolved in a salt solution, e.g., 150 mM sodium phosphate. The polynucleotide or polynucleotide construct may further comprise auxiliary agents, including, but not limited to, poloxamers, DMSO, IGEPAL® CA 630, NONIDET NP-40®, Nonidet P40, Triton X-100™, Triton X-114™, sodium dodecyl sulfate, Tween-20®, Tween-80®, stachyose, EDTA, Thesit®, combinations thereof, and reaction, association, or dissociation products thereof.

In one embodiment, the present invention provides a method of treating or preventing an autoimmune disease or an inflammatory condition, for example, an inflammatory demyelinating disease, comprising administering to a mammal a therapeutically effective amount of a non-infectious, non-integrating polynucleotide or polynucleotide construct comprising a polynucleotide selected from the group consisting of (a) a polynucleotide that hybridizes under stringent conditions to the nucleotide sequence of SEQ ID NO:1 or the complement thereof, wherein the polynucleotide sequence encodes a polypeptide that has anti-viral and/or anti-proliferative activity; (b) a polynucleotide that encodes a polypeptide comprising an amino acid sequence which, except for at least one but not more than 20 amino acid substitutions, deletions, or insertions, is identical to amino acids −21 to 166, 1 to 166, or 2 to 166 in SEQ ID NO:2, wherein the polypeptide has anti-viral and/or anti-proliferative activity; and (c) a polynucleotide encoding an IFNβ or an active fragment or variant thereof. Preferably, the present invention provides a method of treating or preventing an autoimmune disease, and in particular multiple sclerosis.

In an additional embodiment, the present invention provides a method of treating, preventing, or reducing the symptoms of autoimmune diseases and other conditions related to inflammation in a mammal, for example, treating, preventing, or reducing the symptoms an inflammatory demyelinating disease in a mammal comprising administering in vivo into a tissue of a mammal, preferably a mammal in need of such treatment, a composition comprising a polynucleotide or polynucleotide construct encoding interferon-beta (IFNβ), or an active fragment or variant thereof, together with a pharmaceutically acceptable carrier, where the IFNβ-encoding polynucleotide or polynucleotide construct is either a DNA plasmid encoding said IFNβ or active fragment or variant thereof through operable association with a promoter or a messenger RNA, and where the IFNβ-encoding polynucleotide or polynucleotide construct is free from association with liposomal formulations and charged lipids. In this embodiment, the polynucleotide or polynucleotide construct is incorporated into the cells of said mammal, and a therapeutically effective amount of IFNβ, or active fragment or variant thereof is expressed.

In yet another embodiment, the present invention provides a method of treating, preventing, or reducing the symptoms of autoimmune diseases and other conditions related to inflammation in a mammal, for example, treating, preventing, or reducing the symptoms an inflammatory demyelinating disease in a mammal comprising administering in vivo into a tissue of a mammal, preferably a mammal in need of such treatment, a composition comprising a polynucleotide or polynucleotide construct encoding interferon-beta (IFNβ), or an active fragment or variant thereof, together with a pharmaceutically acceptable carrier, where the IFNβ-encoding polynucleotide or polynucleotide construct is either a DNA plasmid encoding said IFNβ or active fragment or variant thereof through operable association with a promoter or a messenger RNA, and where the tissue of administration is either muscle, skin, or blood. In this embodiment, the polynucleotide or polynucleotide construct is incorporated into the cells of said mammal, and a therapeutically effective amount of IFNβ, or active fragment or variant thereof is expressed.

According to the present invention, “polynucleotides encoding interferon-beta (IFNβ), or active fragments or variants thereof” include, but are not limited to: (a) polynucleotides comprising a nucleic acid that hybridizes under stringent conditions to the complement of SEQ ID NO:1, wherein the polynucleotide sequence encodes a polypeptide that has anti-viral or anti-proliferative activity; (b) polynucleotides comprising a nucleic acid that encodes a polypeptide which, except for at least one but not more than 20 individual amino acid substitutions, deletions, or insertions, is identical to amino acids 1 to 166 in SEQ ID NO:2, wherein the polypeptide has anti-viral or anti-proliferative activity; and (c) polynucleotides comprising a nucleic acid that encodes a polypeptide at least 80, 85%, 90%, 91%, 92%, 93%, 94%, 95%, 96%, 97%, 98%, or 99% identical to amino acids 1 to 166 in SEQ ID NO:2, wherein the polypeptide has anti-viral or anti-proliferative activity.

It is to be noted that the term “a” or “an” entity refers to one or more of that entity; for example, “a polynucleotide,” is understood to represent one or more polynucleotides or polynucleotide constructs. As such, the terms “a” (or “an”), “one or more,” and “at least one” can be used interchangeably herein.

As used herein, the term “IFNβ or active fragment or variant thereof” refers to any mammalian IFNβ, fragment or variant. The choice of species will depend largely on the animal being treated. IFNβ has been isolated from a large variety of mammalian species. Examples include, but are not limited to equine IFNβ having the amino acid sequence of SEQ ID NO:16, encoded by the nucleotide sequence SEQ ID NO: 15, porcine IFNβ having the amino acid sequence of SEQ ID NO: 18, encoded by the nucleotide sequence SEQ ID NO: 17, feline IFNβ having the amino acid sequence of SEQ ID NO:20, encoded by the nucleotide sequence SEQ ID NO: 19, rat IFNβ having the amino acid sequence of SEQ ID NO:22, encoded by the nucleotide sequence SEQ ID NO:21, murine IFNβ having the amino acid sequence of SEQ ID NO:4, encoded by the nucleotide sequence SEQ ID NO:3, and human IFNβ having the amino acid sequence of SEQ ID NO:2, encoded by the nucleotide sequence SEQ ID NO:1, and active fragments or variants of any of these IFNβ molecules. For treatment of humans, use of human IFNβ or active fragments or variants thereof, is preferred.

The term “nucleic acid” is intended to encompass a singular “nucleic acid” as well as plural “nucleic acids,” and refers to an isolated molecule or construct, e.g., virus genomes (preferably non-infectious), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide or polynucleotide construct. A nucleic acid may be provided in linear (e.g., mRNA), circular (e.g., plasmid), or branched form as well as double-stranded or single-stranded forms. A nucleic acid may comprise a conventional phosphodiester bond or a non-conventional bond (e.g., an amide bond, such as found in peptide nucleic acids (PNA)).

The term “polynucleotide” refers to any one or more nucleic acid segments or constructs (e.g., DNA or RNA oligomers, mRNA or pDNA). The polynucleotide may be provided in linear, circular (e.g., plasmid), or branched form as well as double-stranded or single-stranded form. The polynucleotide may comprise a conventional phosphodiester bond or a non-conventional bond (e.g., an amide bond, such as found in peptide nucleic acids (PNA)). Two or more polynucleotides of the present invention can be present in a single construct, e.g., on a single plasmid, or in separate constructs, e.g., on separate plasmids. Furthermore, any polynucleotide may encode a single polypeptide, e.g., a single antigen, cytokine, or regulatory polypeptide, or may encode more than one polypeptide, e.g., a polynucleotide may encode two or more polypeptides. In addition, a polynucleotide may encode a regulatory element such as a promoter or a transcription terminator, or may encode a specific element of a polypeptide or protein, such as a secretory signal peptide or a functional domain.

Nucleic acids and/or polynucleotides and/or polynucleotide constructs of the present invention, e.g., plasmid DNA, derivatives of plasmid DNA, mRNA, linear DNA, viral genomes, or polynucleotide fragments contained therein may be formulated into any of the various compositions and may be used in any of the methods disclosed herein. For aqueous compositions used in vivo, use of sterile pyrogen-free water is preferred. Such formulations will contain an effective amount of a polynucleotide or polynucleotide construct together with a suitable salt and/or auxiliary agent as disclosed herein, in order to prepare pharmaceutically acceptable compositions suitable for optimal administration to a vertebrate. Insoluble polynucleotides or polynucleotide constructs may be solubilized in a weak acid or weak base, and then diluted to the desired volume, for example, with an aqueous solution of the present invention. The pH of the solution may be adjusted as appropriate. In addition, a pharmaceutically acceptable additive can be used to provide an appropriate osmolarity. Such additives are within the purview of one skilled in the art.

The amount of a polynucleotide or polynucleotide construct included in a composition of the present invention depends on many factors, including the age and weight of the subject, the delivery method and route, the type of treatment desired, and the type of polynucleotide or polynucleotide construct being administered. In general, a composition of the present invention includes from about 1 ng to about 30 mg of a polynucleotide or polynucleotide construct, more preferably, from about 100 ng to about 10 mg of a polynucleotide or polynucleotide construct.

Certain preferred compositions of the present invention may include about 1 ng of a polynucleotide, about 5 ng of a polynucleotide, about 10 ng of a polynucleotide, about 50 ng of a polynucleotide, about 100 ng of a polynucleotide, about 500 ng of a polynucleotide, about 1 fig of a polynucleotide, about 5 μg of a polynucleotide, about 10 μg of a polynucleotide, about 50 μg of a polynucleotide, about 100 μg of a polynucleotide, about 150 μg of a polynucleotide, about 200 μg of a polynucleotide, about 250 μg of a polynucleotide, about 300 μg of a polynucleotide, about 350 μg of a polynucleotide, about 400 μg of a polynucleotide, about 450 μg of a polynucleotide, about 500 μg of a polynucleotide, about 550 μg of a polynucleotide, about 600 μg of a polynucleotide, about 650 μg of a polynucleotide, about 700 μg of a polynucleotide, about 750 μg of a polynucleotide, about 800 μg of a polynucleotide, about 850 μg of a polynucleotide, about 900 μg of a polynucleotide, about 950 μg of a polynucleotide, about 1 μg of a polynucleotide, about 5 μg of a polynucleotide, about 10 μg of a polynucleotide, about 15 μg of a polynucleotide, about 20 μg of a polynucleotide, about 25 μg of a polynucleotide, and about 30 μg of a polynucleotide.

In one embodiment, a polynucleotide or polynucleotide construct of the present invention is RNA. Preferably in this embodiment, the RNA is in the form of messenger RNA (mRNA). Methods for introducing RNA sequences into vertebrate cells is described in U.S. Pat. No. 5,580,859, the disclosure of which is incorporated herein by reference in its entirety. Methods of expressing IFNβ or active fragments or variants thereof from RNA replicons are disclosed in WO 98/26084.

Alternatively, the RNA is in the form of an RNA virus genome. Preferably an RNA virus genome of the present invention is noninfectious, (i.e., does not result in the production of infectious virus particles in vertebrate cells). Suitable RNA virus genomes include, but are not limited to, alphavirus genomes, picornavirus genomes, and retrovirus genomes. Methods for the in vivo introduction of non-infectious viral genomes to vertebrate tissues are well known to those of ordinary skill in the art and are described, e.g., in Altman-Hamamdzic, S., et al., Gene Therapy 4, 815-822 (1997), in U.S. Pat. No. 4,980,289, Dec. 25, 1990, and in Miller, A. D., et al., Meth. Enzymol. 217:581-599 (1993), the disclosures of which are incorporated herein by reference in their entireties. Viral replicons, i.e., non-infectious RNA virus genomes packaged in a viral coat, e.g., a picornavirus coat or an alphavirus coat, are also useful for efficient administration of RNA. See, e.g., U.S. Pat. No. 5,766,602, U.S. Pat. No. 5,614,413, and PCT Publication No. WO 95/07994, the disclosures of which are incorporated herein by reference in their entireties.

Preferably, the polynucleotide or polynucleotide construct is DNA. In the case of DNA, a polynucleotide or polynucleotide construct encoding a polypeptide is normally operably associated with a promoter. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide or polynucleotide construct to direct cell-specific transcription.

The polynucleotides or polynucleotide construct used in the methods of the present invention may be associated with additional polynucleotides or polynucleotide construct which encode secretory or signal peptides, which direct the secretion of the polypeptide encoded by the polynucleotide or polynucleotide construct of the present invention. Those of ordinary skill in the art are aware that polypeptides secreted by mammalian cells normally have a signal peptide which is cleaved from the complete polypeptide to produce a secreted “mature” form of the polypeptide. In one embodiment, either the native leader sequence of IFNβ is used, or a functional derivative of that sequence that retains the ability to direct the secretion of the peptide that is operably linked to it. Alternatively, a heterologous mammalian leader sequence, or a functional derivative thereof, may be used. For example, the wild-type leader sequence may be substituted with the leader sequence of human tissue plasminogen activator or mouse β-glucuronidase. Additionally, a completely synthetic (i.e., an amino acid sequence not occurring in nature) amino acid coding sequence that functions as a mammalian leader sequence can be constructed by those skilled in the art utilizing recombinant DNA techniques.

The polynucleotide or polynucleotide construct can be an expression vector. A typical mammalian expression vector contains the promoter element, which mediates the initiation of transcription of mRNA, the polypeptide coding sequence, and signals required for the termination of transcription and polyadenylation of thc transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from retroviruses, e.g., RSV, HTLVI, HIVI, MPSV and the immediate early promoter of the cytomegalovirus (CMV IEP). However, cellular elements can also be used (e.g., the human actin promoter, metallothionein promoter). In humans, CMV IEP is preferred. Suitable expression vectors for use in practicing the present invention include, for example, vectors such as PSVL and PMSG (Pharmacia, Uppsala, Sweden), pRSVcat (ATCC 37152), pSV2dhfr (ATCC 37146) and pBC12MI (ATCC 67109), VR1051, VR1055, and pcDNA3 (Invitrogen, San Diego, Calif.). All forms of DNA, whether replicating or non-replicating, which do not become integrated into the genome, and which are expressible, are within the methods contemplated by the invention.

The vector containing the DNA sequence (or the corresponding RNA sequence) which can be used in accordance with the invention can be a eukaryotic expression vector. Techniques for obtaining expression of exogenous DNA or RNA sequences in a host are known. See, for example, Korman, et al., Proc. Nat. Acad. Sci. (USA) 84:2150-2154 (1987).

A “polynucleotide construct” is a polynucleotide molecule that carries genetic information for encoding one or more molecules, preferably, cytokines. The polynucleotide material delivered to the cells in vivo can take any number of forms. It can contain the entire sequence or only a functionally active fragment of a cytokine gene. The polynucleotide construct is assembled out of components where different selectable genes, origins, promoters, introns, 5′ untranslated (UT) sequence, terminators, polyadenylation signals, 3′ UT sequence, and leader peptides, etc. are put together to make the desired vector. The precise nature of the regulatory regions needed for gene expression can vary between species or cell types, but shall in general include, as necessary, 5′ non-transcribing and 5′ non-translating (non-coding) sequences involved with initiation of transcription and translation respectively, such as the TATA box, capping sequence, CAAT sequence, and the like, with those elements necessary for the promoter sequence being provided by the promoters of the invention. Such transcriptional control sequences can also include enhancer sequences or upstream activator sequences, as desired.

“Non-infectious” means that the polynucleotide or polynucleotide construct does not infect mammalian cells. Specifically, a non-infectious, without more, is not capable of eliciting the production of infectious virus particles which can go on to infect sister cells. Thus, a non-infectious polynucleotide or polynucleotide construct can contain functional sequences from non-mammalian (e.g., viral or bacterial) species, but does not contain non-mammalian, e.g., viral nucleotide sequences which are necessary and sufficient to allow viral replication, capsid formation, packaging and in some cases, envelopment, required to produce infectious virion particles, thus facilitating infection of the construct into additional mammalian cells.

“Non-integrating” means that the polynucleotide or polynucleotide construct does not functionally integrate into the genome of mammalian cells. The construct can be a non-replicating DNA sequence, or specific replicating sequences genetically engineered to lack the ability to integrate into the genome. A non-integrating polynucleotide or polynucleotide construct does not contain functional sequences that facilitate integration of the polynucleotide or polynucleotide construct into the genome of mammalian cells. It is well understood by those of ordinary skill in the art that any polynucleotide or polynucleotide construct, including a “non-integrating” polynucleotide of the present invention may, under very rare circumstances, non-specifically integrate into a mammalian chromosome.

The choice of polynucleotide form depends in part on the desired kinetics and duration of expression. When long-term expression of the polypeptide encoded by the polynucleotide is desired, the preferred form is DNA, preferably plasmid DNA. Alternatively, when short-term expression of the polypeptide encoded by the polynucleotide is desired, the preferred form is RNA, preferably messenger RNA, since RNA is rapidly translated into polypeptide, but is degraded more quickly than DNA.

An operable association is when a polynucleotide encoding a gene product, e.g., a polypeptide, is associated with one or more regulatory sequences in such a way as to place expression of the molecule under the influence or control of the regulatory sequence(s). Two DNA fragments (such as a polypeptide-coding polynucleotide and a promoter associated with the 5′ end of the polynucleotide) are “operably associated” if induction of promoter function results in the transcription of mRNA encoding the desired gene product and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a polynucleotide encoding a polypeptide if the promoter was capable of effecting transcription of that polynucleotide.

A variety of transcription control regions are known to those skilled in the art. Preferred transcription control regions include those which function in vertebrate cells, such as, but not limited to, promoter and enhancer segments from cytomegaloviruses (preferably the immediate early promoter, preferably in conjunction with intron-A), simian virus 40 (preferably the early promoter), retroviruses (such as Rous sarcoma virus), and picornaviruses (particularly an internal ribosome entry site, or IRES, also referred to as a CITE sequence). Other preferred transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Preferably, a DNA polynucleotide or polynucleotide construct of the present invention is part of a circular or linearized plasmid which is preferably non-infectious (i.e., does not result in the production of infectious virus particles in vertebrate cells), and noninitegrating (i.e., does not integrate into the genome of vertebrate cells). A linearized plasmid is a plasmid that was previously circular but has been linearized, for example, by digestion with a restriction endonuclease.

Alternatively, DNA virus genomes may be used to administer DNA polynucleotides or polynucleotide constructs into vertebrate cells. Preferably a DNA virus genome of the present invention is noninfectious, (i.e., does not result in the production of infectious virus particles in vertebrate cells), and nonintegrating (i.e., does not integrate into the genome of vertebrate cells). Suitable DNA virus genomes include herpesvirus genomes, adenovirus genomes, adeno-associated virus genomes, and poxyirus genomes. References citing methods for the in vivo introduction of non-infectious virus genomes to vertebrate tissues are well known to those of ordinary skill in the art, and are cited supra.

“Stringent hybridization conditions” are those experimental parameters that allow an individual skilled in the art to identify similarities between heterologous nucleic acid molecules. See, for example, Sambrook, et al., Molecular Cloning: A Laboratory Manual, Cold Spring Harbor Labs Press, Cold Spring Harbor, N.Y. (1989), and Meinkoth, et al., Anal. Biochem. 138:267-284 (1984), both of which are incorporated herein by reference.

The determination of stringent hybridization conditions involves the manipulation of a set of variables, including ionic strength (M, in moles/liter), the hybridization temperature (° C.), the concentration of helix destabilizing agents (such as formamide), the average length of the shortest chain in the duplex (n), and the percent G+C composition of the fragments being hybridized. For nucleic acid molecules longer than about 50 nucleotides, these variables are inserted into a standard formula to calculate the melting temperature, or Tₘ, of a given nucleic acid molecule, which is the temperature at which two complementary nucleic acid molecule strands will disassociate, assuming 100% complementarity between the two strands:

Tₘ=81.5° C.+16.6 log M+0.41(% G+C)−500/n−0.61(%formamide).

For nucleic acid molecules smaller than about 50 nucleotides, hybrid stability is defined by the dissociation temperature (T_{d}), the temperature at which 50% of the duplexes dissociate. For these smaller molecules, the stability, at a standard ionic strength, is defined by the following equation:

T_{d}=4(G+C)+2(A+T).

A temperature of 5° C. below T_{d} is used to detect hybridization between perfectly matched molecules.

It is also well known by those skilled in the art how base-pair mismatch will affect Tₘ or T_{d} for nucleic acid molecules of different sizes. For example, Tₘ decreases about 1° C. for each 1% of mismatched base-pairs for hybrids greater than about 150 base pairs (bp), and T_{d} decreases about 5° C. for each mispaired base-pair for hybrids below about 50 bp. Conditions for hybrids between about 50 and about 150 base-pairs can be determined empirically. This allows one skilled in the art to set the hybridization conditions (by altering, for example, the salt concentration, the formamide concentration or the temperature) such that only hybrids with greater than a specified % base-pair mismatch will hybridize. Stringent hybridization conditions are commonly understood by those skilled in the art to be those experimental conditions that will allow no more than about 3-5% base-pair mismatch (i.e., about 95-97% identity between the hybrid strands).

“Stringent conditions” for a hybridization probe larger than 100 nucleotides, for example, may comprise hybridization by overnight incubation at 42° C. in a solution comprising: 50% formamide, 5× SSC (750 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by repeatedly washing the filters (at least three times) in 0.1× SSC and 0.1% sodium dodecyl sulfate (w/v) for 20 minutes at about 65° C. Using the formula described above with a probe of about 1000 nucleotides, having an about 40% G+C content, the Tm of fully complementary hybrids will be about 67° C. Thus, the stringent wash at 65° C. will allow detection of hybrids having 2% or less base-pair mismatch.

By a polynucleotide or polynucleotide construct encoding a polypeptide at least, for example, 95% “identical” to an amino acid sequence of a reference IFNβ polypeptide is intended that the amino acid sequence of the polypeptide is identical to the reference sequence except that the polypeptide sequence may include up to five amino acid alterations per each 100 amino acids of the reference amino acid sequence. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference amino acid sequence.

As used herein, an IFNβ-encoding polynucleotide or polynucleotide construct refers to any polynucleotide encoding a polypeptide with IFNβ activity, i.e., it refers generally to a polynucleotide or polynucleotide construct which encodes IFNβ and also to polynucleotides or polynucleotide constructs encoding active fragments or active variants of IFNβ. As used herein, the term “polypeptide” is intended to encompass a singular “polypeptide” as well as plural “polypeptides,” and comprises any chain or chains of two or more amino acids. Thus, as used herein, the terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

As a practical matter, whether any particular polypeptide is at least 80%, 85%, 90%, 92%, 95%, 96%, 97%, 98% or 99% identical to, for instance, the amino acid sequence depicted herein as SEQ ID NO:2, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

It will be recognized in the art that some amino acid sequences of the polypeptides described herein can be varied without significant effect on the functional activity of the polypeptides. If such differences in sequence are contemplated, it should be remembered that there will be critical areas on the polypeptide which determine activity. Such variations include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” (Science 247:1306-1310 (1990)). Compositions within the scope of the invention can be assayed according to the antiproliferation assay described herein. Amino acids that are critical for cytokine activity can also be determined by structural analysis such as crystallization, nuclear magnetic resonance or photoaffinity labeling (Smith, et al., J. Mol. Biol. 224:899-904 (1992) and de Vos, et al. Science 255:306-312 (1992)).

The present invention further relates to using variants of IFNβ-encoding polynucleotides or polynucleotide constructs, which encode portions, analogs or derivatives of IFNβ. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Alterations in the coding regions may produce conservative or non-conservative amino acid substitutions, deletions or additions. Especially preferred among these are silent substitutions, additions and deletions, which do not alter the basic properties and activities of the cytokine or portions thereof. Also especially preferred in this regard are conservative substitutions. For example, aromatic amino acids that can be conservatively substituted for one another include phenylalanine, tryptophan, and tyrosine. Hydrophobic amino acids that can be conservatively substituted for one another include leucine, isoleucine, and valine. Polar amino acids that can be conservatively substituted for one another include glutamine and asparagine. Basic amino acids that can be conservatively substituted for one another include arginine, lysine, and histidine. Acidic amino acids that can be conservatively substituted for one another include aspartic acid and glutamic acid. Small amino acids that can be conservatively substituted for one another include alanine, serine, threonine, methionine, and glycine.

Substitutions, deletions, or insertions can be made outside of the region encoding the shortest active fragment of IFNβ, without affecting the activity of the cytokine. Further, mutated proteins (or muteins) often retain a biological activity that is similar to that of the naturally occurring protein. For example, Gayle and coworkers (J. Biol. Chem. 268. 22105-22111 (1993)) conducted an extensive mutational analysis of the human cytokine IL-1α. They used random mutagenesis to generate over 3,500 individual IL-1α mutants with an average of 2.5 amino acid changes per mutein over the entire length of the molecule. Multiple mutations were examined at every possible amino acid and, on average, each mutein's amino acid sequence was 98.4% identical to that of naturally occurring IL-1α. The investigators observed that most of the molecule could be mutated with little effect on either binding or biological activity, and that 75% of the molecule may not contribute significantly to the biological activity of the molecule.

Similarly, Gronenborn and colleagues (FEBS Letters 231: 135-138 (1988)) analyzed the receptor binding activity of six mutant IL-1α polypeptides. Each mutant contained a single amino acid alteration from the naturally occurring IL-1α polypeptide and was examined under four sets of experimental conditions. In this study, the investigators found very little difference between the receptor binding activity of the mutants and naturally occurring IL-1α.

Further, Zurawski and colleagues (EMBO J. 12: 5113-5119 (1993)) studied residues 41-142 of mIL-2 by generating 1,090 muteins. The extent of the mutagenesis was such that there was an average of 11 different amino acid substitutions per naturally occurring amino acid residue, with the exception of the extreme N- and C-termini and residues 31-40. The mIL-2 muteins were assayed for specific activity and compared to that of naturally occurring mIL-2. The degree to which the specific activity was antagonized by a previously characterized mIL-2 mutant was also assessed. The investigators observed that in the 149 residue mIL-2 protein, only 23 residues are important for interaction with IL-2R, 18 residues are presumed to be part of the structural core, and three additional residues are important for structure. 98 mIL-2 residues (or 65% of the protein) were assigned as relatively unimportant residues.

Thus, a polynucleotide sequence encoding a polypeptide of the present invention can encode a polypeptide having one to twenty amino acid substitutions, deletions or insertions, either from natural mutations or human manipulation, relative to the full length or mature IFNβ. By “amino acid substitutions, deletions or insertions” is meant that single, individual amino acids are substituted, deleted and/or inserted. Preferably, no more than one to fifteen substitutions, deletions or insertions are present, relative to the full length or mature IFNβ (excluding the signal sequence). More preferably, no more than one to ten substitutions, deletions or insertions are present. Still more preferably, no more than one to five substitutions, deletions or insertions are present.

Further, mutated forms of IFNβ (or muteins) often retain a biological activity that is similar to that of the naturally occurring protein. For example, Whitty and coworkers (Biochemistry 39:2538-2551 (2000)) conducted a systematic structure-based mutational analysis of the human cytokine IFNβ-1α. They used alanine scanning mutagenesis to generate 15 individual IFNβ mutants with an average of 2-8 contiguous amino acid changes per mutein over the entire surface-exposed length of the molecule. Altogether 65 of a total of 166 amino acids residues were mutated in this study. The investigators observed that the following regions were critical for IFNβ biological activity: residues 15-42, 71-73, 130-139, and 150-160. Residues outside these regions could be mutated without affecting the biological activity of human IFNβ.

By “active fragment or variant” is intended a fragment or variant of IFNβthat displays similar or enhanced anti-viral and/or anti-proliferative activity as the mature or full length cytokine. For example, a full length hIFNβ is set forth in amino acids −21 to 166 of SEQ ID NO:2, with mature forms being amino acids 1 to 166, or 2 to 166 in SEQ ID NO:2. Active fragments and/or variants of hIFNβ include, but are not limited to polypeptides comprising amino acids −21 to 166, 1 to 166, or 2 to 166 in SEQ ID NO:2, wherein the valine at position 101 is substituted with phenylalanine, tyrosine, tryptophan, or histidine. Other suitable IFNβ fragments or variants are disclosed in Runkel, et al., Biochemistry 39:2538-2511 (2000); U.S. Pat. No. 6,127,332, and WO 98/27211, which are herein incorporated by reference.

Assays of anti-viral and/or anti-proliferative activity in vitro are well known to those of ordinary skill in the art. An example is shown in the Examples section, infra.

Other therapies for autoimmune disease, e.g. MS, can be used in conjunction with the present invention. Active agents contemplated for use are synthetic or natural compounds which demonstrate a biological effect when introduced into a living creature and include peptides, small molecules, carbohydrates, nucleic acids, and proteins. Proteins contemplated for use include potent cytokines, including various hematopoietic factors such as granulocyte-colony stimulating factor (G-CSF), kerantinocyte growth factor (KGF), stem cell factor (SCF), megakaryocyte growth differentiation factor (MGDF), granulocyte macrophage-colony stimulating factor (GM-CSF), the interferons (alpha, and beta), the interleukins (2-12), erythropoietin (EPO), fibroblast growth factor (FGF), stem cell factor (SCF), nerve growth factor (NGF), brain-derived neurotrophic factor (BDNF), neurotrophic factor-3 (NT3), platelet-derived growth factor (PDGF), tumor growth factor (alpha, beta), interleukin-1 receptor antagonist (IL-1ra), osteoprotegerin (OPG), glial cell line derived neurotrophic factor (GDNF), p38 inhibitors and obesity protein (OB protein).

A polynucleotide, polynucleotide construct, or composition comprising a polynucleotide or polynucleotide construct can be administered prior to the commencement of one or more of the additional therapies, during the practice of one or more of the additional therapies, and after the end of one or more of the additional therapies.

For the methods of the present invention, a single polynucleotide, polynucleotide construct, or composition comprising a polynucleotide or polynucleotide construct containing more than one polynucleotide sequence encoding one or more molecules may be administered. Alternatively, more than one polynucleotide, polynucleotide construct, or composition comprising a polynucleotide or polynucleotide construct, each containing polynucleotide sequences encoding one or more molecules may be co-injected or sequentially injected. For example, a single polynucleotide or polynucleotide construct containing one polynucleotide encoding IFNβ or an active fragment or variant thereof and another polynucleotide encoding an additional cytokine or a therapeutic molecule can be injected. Alternatively, two polynucleotides or polynucleotide constructs can be injected where one encodes an IFNβ or an active fragment or variant thereof, and the other encodes another cytokine or a therapeutic molecule. For example, an IFNβ-expressing polynucleotide or polynucleotide construct can be co-injected with a polynucleotide or polynucleotide construct encoding a different cytokine.

The term “cytokine” refers to polypeptides, including but not limited to interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, and IL-18), α interferons (e.g, IFNα), β interferons (e.g., IFNβ), γ interferons (e.g., IFNγ), ω interferon (IFN ω), τ interferons (IFNτ), colony stimulating factors (CSFS, e.g., CSF-1, CSF-2, and CSF-3), granulocyte-macrophage colony stimulating factor (GMCSF), epidermal growth factor (EGF), fibroblast growth factors (FGFs, e.g., acidic fibroblast growth factor, basic fibroblast growth factor, FGF-1, FGF-2, FGF-3, FGF-4, and FGF-5), transforming growth factor (TGF, e.g., TGFα and TGFβ), platelet-derived growth factor (PDGF), tumor necrosis factors (TNFs, e.g., TNF-α and TNF-β), and insulin-like growth factors (IGFs, e.g, IGF-I and IGF-II).

If the polynucleotide or polynucleotide construct of the present invention is administered as a pharmaceutical composition, the pharmaceutical composition can be formulated according to known methods for preparing pharmaceutical compositions, whereby the substance to be delivered is combined with a pharmaceutically acceptable carrier vehicle. Suitable vehicles and their preparation are described, for example, in Remington's Pharmaceutical Sciences, 16^{th} Edition, A. Osol, Ed., Mack Publishing Co., Easton, Pa. (1980), and Remington's Pharmaceutical Sciences, 19^{th} Edition, A. R. Gennaro, Ed., Mack Publishing Co., Easton, Pa. (1995).

Transfection Facilitating Agents

Compositions of the present invention can also include one or more transfection facilitating materials that facilitate delivery of polynucleotides or polynucleotide constructs to the interior of a cell, and/or to a desired location within a cell. Examples of the transfection facilitating materials include, but are not limited to lipids, preferably cationic lipids; inorganic materials such as calcium phosphate, and metal (e.g., gold or tungsten) particles (e.g., “powder” type delivery solutions); peptides, including cationic peptides, targeting peptides for selective delivery to certain cells or intracellular organelles such as the nucleus or nucleolus, and amphipathic peptides, i.e. helix forming or pore forming peptides; basic proteins, such as histories; asialoproteins; viral proteins (e.g., Sendai virus coat protein); pore-forming proteins; and polymers, including dendrimers, star-polymers, “homogenous” poly-amino acids (e.g., poly-lysine, poly-arginine), “heterogenous” poly-amino acids (e.g., mixtures of lysine & glycine), co-polymers, polyvinylpyrrolidinone (PVP), and polyethylene glycol (PEG). Furthermore, those auxiliary agents of the present invention which facilitate and enhance the entry of a polynucleotide or polynucleotide construct into vertebrate cells in vivo, may also be considered “transfection facilitating materials.”

Certain embodiments of the present invention may include lipids as a transfection facilitating material, including cationic lipids (e.g., DMRIE, DOSPA, DC-Chol, GAP-DLRIE), basic lipids (e.g., steryl amine), neutral lipids (e.g., cholesterol), anionic lipids (e.g., phosphatidyl serine), and zwitterionic lipids (e.g., DOPE, DOPC).

Examples of cationic lipids are 5-carboxyspermylglycine dioctadecylamide (DOGS) and dipalmitoyl-phophatidylethanolamine-5-carboxyspermylamide (DPPES). Cationic cholesterol derivatives are also useful, including {3β-[N-N′,N′-dimethylamino)ethane]-carbomoyl}-cholesterol (DC-Chol). Dimethyldioctdecyl-ammonium bromide (DDAB), N-(3-aminopropyl)-N,N-(bis-(2-tetradecyloxyethyl))-N-methyl-ammonium bromide (PADEMO), N-(3-aminopropyl)-N,N-(bis-(2-dodecyloxyethyl))-N-methyl-ammonium bromide (PADELO), N,N,N-tris-(2-dodecyloxy)ethyl-N-(3-amino)propyl-ammonium bromide (PATELO), and N¹-(3-aminopropyl)((2-dodecyloxy)ethyl)-N²-(2-dodecyloxy)ethyl-1-piperazinaminium bromide (GALOE-BP) can also be employed in the present invention.

Non-diether cationic lipids, such as DL-1,2-dioleoyl-3-dimethylaminopropyl-β-hydroxyethylammonium (DORI diester), 1-O-oleyl-2-oleoyl-3-dimethylaminopropyl-β-hydroxyethylammonium (DORI ester/ether), and their salts promote in vivo gene delivery. Preferred cationic lipids comprise groups attached via a heteroatom attached to the quaternary ammonium moiety in the head group. A glycyl spacer can connect the linker to the hydroxyl group.

Preferred cationic lipids for use in certain embodiments of the present invention include DMRIE ((±)-N-(2-hydroxyethyl)-N,N-dimethyl-2,3-bis(tetradecyloxy)-1-propanaminium bromide), and GAP-DMORIE ((+)-N-(3-aminopropyl)-N,N-dimethyl-2,3-bis(syn-9-tetradeceneyloxy)-1-propanaminium bromide).

Also preferred are (±)-N,N-dimethyl-N-[2-(sperminecarboxamido)ethyl]-2,3-bis(dioleyloxy)-1-propaniminium pentahydrochloride (DOSPA), (±)-N-(2-aminoethyl)-N,N-dimethyl-2,3-bis(tetradecyloxy)-1-propaniminium bromide (β-aminoethyl-DMRIE or βAE-DMRIE) (Wheeler, et al., Biochim. Biophys. Acta 1280:1-11 (1996)), and (±)-N-(3-aminopropyl)-N,N-dimethyl-2,3-bis(dodecyloxy)-1-propaniminium bromide (GAP-DLRIE) (Wheeler, et al., Proc. Natl. Acad. Sci. USA 93:11454-11459 (1996)), which have been developed from DMRIE.

Other examples of DMRIE-derived cationic lipids that are useful for the present invention are (±)-N-(3-aminopropyl)-N,N-dimethyl-2,3-(bis-decyloxy)-1-propanaminium bromide (GAP-DDRIE), (±)-N-(3-aminopropyl)-N,N-dimethyl-2,3-(bis-tetradecyloxy)-1-propanaminium bromide (GAP-DMRIE), (±)-N-((N″-methyl)-N′-ureyl)propyl-N,N-dimethyl-2,3-bis(tetradecyloxy)-1-propanaminium bromide (GMU-DMRIE), (±)-N-(2-hydroxyethyl)-N,N-dimethyl-2,3-bis(dodecyloxy)-1-propanaminium bromide (DLRIE), and (±)-N-(2-hydroxyethyl)-N,N-dimethyl-2,3-bis-([Z]-9-octadecenyloxy)propyl-1-propaniminium bromide (HP-DORIE).

A preferred cationic lipid of the present invention is a “cytofectin.” As used herein, a “cytofectin” refers to a subset of cationic lipids which incorporate certain structural features including, but not limited to, a quaternary ammonium group and/or a hydrophobic region (usually with two or more alkyl chains), but which do not require amine protonation to develop a positive charge. Examples of cytofectins may be found, for example, in U.S. Pat. No. 5,861,397, which is incorporated herein by reference in its entirety.

Preferred cytofectins for use in the present invention, include DMRIE ((±)-N-(2-hydroxyethyl)-N,N-dimethyl-2,3-bis(tetradecyloxy)-1-propanaminium bromide), GAP-DMORIE ((±)-N-(3-aminopropyl)-N,N-dimethyl-2,3-bis(syn-9-tetradeceneyloxy)-1-propanaminium bromide), and GAP-DLRIE ((±)-N-(3-aminopropyl)-N,N-dimethyl-2,3-(bis-dodecyloxy)-1-propanaminium bromide).

Preferably, the cationic lipid is mixed with one or more co-lipids. For purposes of definition, the term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films. A preferred class of co-lipids are the zwitterionic phospholipids, which include the phosphatidylethanolamines and the phosphatidylcholines. Most preferably, the co-lipids are phosphatidylethanolamines, such as, for example, DOPE, DMPE and DPyPE. DOPE and DPyPE are particularly preferred. For immunization, the most preferred co-lipid is DPyPE, which comprises two phytanoyl substituents incorporated into the diacylphosphatidylethanolamine skeleton.

The preferred cationic lipid:co-lipid molar ratio of the present invention is from about 9:1 to about 1:9. More preferably, the cationic lipid:co-lipid molar ratio is from about 4:1 to about 1:4 and, still more preferably, is from about 2:1 to about 1:2. A most preferred cationic lipid:co-lipid molar ratio is about 1:1.

In order to maximize homogeneity, the cationic lipid and co-lipid components of the present invention are preferably dissolved in a solvent such as chloroform, followed by evaporation of the cationic lipid/co-lipid solution under vacuum to dryness as a film on the inner surface of a glass vessel (e.g., a Rotovap round-bottomed flask). Upon suspension in an aqueous solvent, the amphipathic lipid component molecules self-assemble into homogenous lipid vesicles. These lipid vesicles may subsequently be processed to have a selected mean diameter of uniform size prior to complexing with, for example, plasmid DNA according to methods known to those skilled in the art. For example, the sonication of a lipid solution is described in Felgner, P. L., et al., Proc. Natl. Acad. Sci. USA 84:7413-7417 (1987) and in U.S. Pat. No. 5,264,618, the disclosures of which are incorporated herein by reference in their entireties.

In the embodiments including cationic lipids, the polynucleotide or polynucleotide construct(s) are combined with lipids by mixing, for example, a plasmid DNA solution and a solution of cationic lipid:co-lipid liposomes. Preferably, the concentration of each of the constituent solutions is adjusted prior to mixing such that the desired final plasmid DNA/cationic lipid:co-lipid ratio and the desired plasmid DNA final concentration will be obtained upon mixing the two solutions. For example, if the desired final solution is to be 2.5 mM sodium phosphate, the various components of the composition, e.g., plasmid DNA, cationic lipid:co-lipid liposomes, and any other desired auxiliary agents, transfection facilitating materials, or additives are each prepared in 2.5 mM sodium phosphate and then simply mixed to afford the desired complex.

Alternatively, if the desired final solution is to be, e.g., 2.5 mM sodium phosphate, certain components of the composition, e.g., the auxiliary agent and/or cationic lipid:co-lipid liposomes, is prepared in a volume of water which is less than that of the final volume of the composition, and certain other components of the composition, e.g., the plasmid DNA, is prepared in a solution of sodium phosphate at a higher concentration than 2.5 mM, in a volume such that when the components in water are added to the components in the sodium phosphate solution, the final composition is in an aqueous solution of 2.5 mM sodium phosphate. For example, the plasmid DNA could be prepared in 5.0 mM sodium phosphate at one half the final volume, the auxiliary agent and/or cationic lipid:co-lipid liposome is prepared in water at one half the final volume, and then these two elements are mixed together to produce the final composition.

The cationic lipid:co-lipid liposomes are preferably prepared by hydrating a thin film of the mixed lipid materials in an appropriate volume of aqueous solvent by vortex mixing at ambient temperatures for about 1 minute. The thin films are prepared by admixing chloroform solutions of the individual components to afford a desired molar solute ratio followed by aliquoting the desired volume of the solutions into a suitable container. The solvent is removed by evaporation, first with a stream of dry, inert gas (e.g. argon) followed by high vacuum treatment.

A transfection facilitating material can be used alone or in combination with one or more other transfection facilitating materials. Two or more transfection facilitating materials can be combined by chemical bonding (e.g, covalent and ionic such as in lipidated polylysine, PEGylated polylysine) (Toncheva, V., et al., Biochim. Biophys. Acta 1380(3):354-368 (1998)), mechanical mixing (e.g., free moving materials in liquid or solid phase such as “polylysine+cationic lipids”) (Gao, X., and Huang, L., Biochemistry 35:1027-1036 (1996); Trubetskoy, V. S., et al., Biochem. Biophys. Acta 1131:311-313 (1992)), and aggregation (e.g., co-precipitation, gel forming such as in cationic lipids+poly-lactide co-galactide, and polylysine+gelatin).

Other hydrophobic and amphiphilic additives, such as, for example, sterols, fatty acids, gangliosides, glycolipids, lipopeptides, liposaccharides, neobees, niosomes, prostaglandins and sphingolipids, may also be included in the compositions of the present invention. In such compositions, these additives may be included in an amount between about 0.1 mol % and about 99.9 mol % (relative to total lipid). Preferably, these additives comprise about 1-50 mol % and, most preferably, about 2-25 mol %. Preferred additives include lipopeptides, liposaccharides and steroids.

Pharmaceutical Compositions

The pharmaceutical composition can be in the form of an emulsion, gel, solution, suspension, or other form known in the art. In addition, the pharmaceutical composition can also contain pharmaceutically acceptable additives including, for example, diluents, binders, stabilizers, and preservatives. Administration of pharmaceutically acceptable salts of the polynucleotides described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like.

For aqueous pharmaceutical compositions used in vivo, sterile pyrogen-free water is preferred. Such formulations will contain an effective amount of the substance together with a suitable amount of vehicle in order to prepare pharmaceutically acceptable compositions suitable for administration to a human or animal. Insoluble polynucleotides or polynucleotide constructs may be solubilized in a weak acid or weak base, and then diluted to the desired volume, for example, with an aqueous solution of the present invention. The pH of the solution may be adjusted as appropriate. In addition, a pharmaceutically acceptable additive can be used to provide an appropriate osmolarity. Such additives are within the purview of one skilled in the art.

As used herein a “salt” is a substance produced from the reaction between acids and bases which comprises a metal (cation) and a nonmetal (anion). For example, the base M—OH can be combined with the acid H—X to produce the salt M—X+H₂O. Salts can be “acid,” i.e., having one or more unreplaced H atoms from the acid, e.g., NaH2PO₄, “basic,” i.e., containing unreplaced hydroxyl radicals of the base, e.g., Bi(OH)Cl₂, or mixed, i.e., containing two or more metals, e.g., NaKHPO₄. A net neutral valency is maintained between the cationic moiety and the anionic moiety. Salt crystals may be “hydrated” i.e., contain one or more water molecules. Such hydrated salts, when dissolved in an aqueous solution at a ceratin molar concentration, are equivalent to the corresponding anhydrous salt dissolved in an aqueous solution at the same molar concentration. For the present invention, salts which are readily soluble in an aqueous solution are preferred.

The terms “saline” or “normal saline” as used herein refer to an aqueous solution of about 145 mM to about 155 mM sodium chloride, preferably about 154 mM sodium chloride. The terms “phosphate buffered saline” or PBS” refer to an aqueous solution of about 145 mM to about 155 mM sodium chloride, preferably about 154 sodium chloride, and about 10 mM sodium phosphate, at a pH ranging from about 6.0 to 8.0, preferably at a pH ranging from about 6.5 to about 7.5, most preferably at pH 7.2.

Certain embodiments of the present invention are drawn to pharmaceutical compositions comprising a polynucleotide or polynucleotide construct encoding IFNβ, or active fragment or variant thereof, where the polynucleotide is dissolved in a salt solution which improves entry of the polynucleotide or polynucleotide construct into vertebrate cells in vivo. Preferred salts in which to dissolve a polynucleotide or polynucleotide construct encoding IFNβ, or active fragment or variant thereof, include but are not limited to sodium phosphate, sodium acetate, sodium bicarbonate, sodium sulfate, sodium pyruvate, potassium phosphate, potassium acetate, potassium bicarbonate, potassium sulfate, potassium pyruvate, disodium DL-α-glycerol-phosphate, and disodium glucose-6-phosphate. “Phosphate” salts of sodium or potassium can be either the monobasic form, e.g., NaHPO₄, or the dibasic form, e.g., Na₂HPO₄, but a mixture of the two, resulting in a desired pH, is most preferred. The most preferred salts are sodium phosphate or potassium phosphate. As used herein, the terms “sodium phosphate” or “potassium phosphate,” refer to a mixture of the dibasic and monobasic forms of each salt to present at a given pH.

Salts of the present invention are preferably dissolved in aqueous solution at concentrations which enhance entry of an IFNβ encoding polynucleotide or polynucleotide construct, or an active fragment or variant thereof, into vertebrate cells in vivo, and/or enhance polypeptide expression, relative to saline, PBS, or water. For example, in certain embodiments, a polynucleotide or polynucleotide construct encoding IFNβ or an active fragment or variant thereof is dissolved in a salt solution of about 150 mM NaHPO₄, Na₂HPO₄, or NaHCO₃.

Additional embodiments of the present invention are drawn to pharmaceutical compositions comprising a polynucleotide or polynucleotide construct encoding IFNβ, or an active fragment or variant thereof, and an auxiliary agent. The present invention is further drawn to methods to use such compositions, methods to make such compositions, and pharmaceutical kits. As used herein, an “auxiliary agent” is a substance included in a composition for its ability to enhance, relative to a composition which is identical except for the inclusion of the auxiliary agent, the entry of polynucleotides or polynucleotide constructs into vertebrate cells in vivo, and/or the in vivo expression of polypeptides encoded by such polynucleotides or polynucleotide constructs. Auxiliary agents of the present invention include nonionic, anionic, cationic, or zwitterionic surfactant or deteregents, with nonionic, anionic, cationic, or zwitterionic surfactant or detergents, with nonionic surfactant or detergents being preferred, chelators, Dnase inhibitors, agents that aggregate or condense nucleic acids, emulsifying or solubilizing agents, wetting agents, gel-forming agents, and buffers.

Auxiliary Agents

Preferred auxiliary agents of the present invention include non-ionic detergents and surfactant such as poloxaners. Poloxamers are a series of non-ionic surfactant that are block copolymers of ethylene oxide and propylene oxide. The poly(oxyethylene) segment is hydrophillic and the poly(oxypropylene) segment is hydrophobic. The physical forms are liquids, pastes or solids. The molecular weight ranges from 1000 to greater than 16000. The basic structure of a poloxaner is HO—(CH₂CH₂O)ₓ—[CH₂CHO(CH₃)]_{y}—(CH₂CH₂O)ₓ—H, where x and y represent repeating units of ethylene oxide and propylene oxide respectively. Thus, the propylene oxide (PO) segment is sandwiched between two ethylene oxide (EO) segments, (EO—PO—EO). The number of x's and y's distinguishes individual poloxamers. If the ethylene oxide segment is sandwiched between two propylene oxide segments, (PO—EO—PO), then the resulting structure is a reverse poloxaner. The basic structure of a reverse poloxamer is HO—[CH(CH₃)CH₂O)ₓ]—(CH₂CH₂O)_{y}—[CH₂CHO(CH₃)]ₓ—H.

Poloxmers of the present invention include, but are not limited to commercially available poloxamers such as Pluronic® L121 (ave. MW:4400), Pluronic® L101 (ave. MW:3800), Pluronic® L81 (ave. MW:2750), Pluronic® L61 (ave. MW:2000), Pluronic® L31 (ave. MW: 1100), Pluronic® L122 (ave. MW:5000), Pluronic® L92 (ave. MW:3650), Pluronic® L72 (ave. MW:2750), Pluronic® L62 (ave. MW:2500), Pluronic® L42 (ave. MW:1630), Pluronic® L63 (ave. MW:2650), Pluronic® L43 (ave. MW: 1850), Pluronic® L64 (ave. MW:2900), Pluronic® L44 (ave. MW:2200), Pluronic® L35 (ave. MW:1900), Pluronic® P123 (ave. MW:5750), Pluronic® P103 (ave. MW:4950), Pluronic® P104 (ave. MW:5900),Pluronic® P84 (ave. MW:4200),Pluronic® P105 (ave. MW:6500), Pluronic® P85 (ave. MW:4600), Pluronic® P75 (ave. MW:4150), Pluronic® P65 (ave. MW:3400), Pluronic® F127 (ave. MW: 12600), Pluronic® F98 (ave. MW: 13000), Pluronic® F87 (ave. MW:7700), Pluronic® F77 (ave. MW:6600), Pluronic® F 108 (ave. MW: 14600), Pluronic® F98 (ave. MW: 13000), Pluronic® F88 (ave. MW:11400), Pluronic® F68 (ave. MW:8400), and Pluronic® F38 (ave. MW:4700).

Reverse poloxamers of the present invention include, but are not limited to Pluronic® R31R1 (ave. MW:3250), Pluronic® R 25R1 (ave. MW:2700), Pluronic® R17R1 (ave. MW:1900), Pluronic® R31R2 (ave. MW:3300), Pluronic® R25R2 (ave. MW:3100), Pluronic® R17R2 (ave. MW:2150), Pluronic® R12R3 (ave. MW:1800),Pluronic® R31R4 (ave. MW:4150), Pluronic® R25R4 (ave. MW:3600), Pluronic® R22R4 (ave. MW:3350), Pluronic® R17R4 (ave. MW:3650), Pluronic® R25R5 (ave. MW:4320), Pluronic® R10R5 (ave. MW:1950), Pluronic® R25R8 (ave. MW:8850), Pluronic® R17R8 (ave. MW:7000), Pluronic® R10R8 (ave. MW:4550).

Other commercially available poloxamers include compounds that are block copolymer of polyethylene and polypropylene glycol such as Synperonic® L121, Synperonic® L122, Synperonic® P104, Synperonic® P105, Synperonic® P123, Synperonic® P85, and Synperonic® P94; and compounds that are nonylphenyl polyethylene glycol such as Synperonic® NP10, Synperonic® NP30, and Synperonic® NP5.

Preferred auxiliary agents include non-ionic detergents and surfactants such as Pluronic® F68, Pluronic® F77, Pluronic® F108, Pluronic® F127, Pluronic® P65, Pluronic® P85, Pluronic® P103, Pluronic® P104, Pluronic® P105, Pluronic® P123, Pluronic® L31, Pluronic® L43, Pluronic® L44, Pluronic® L61, Pluronic® L62, Pluronic® L64, Pluronic® L81, Pluronic® L92, Pluronic® L101, Pluronic® L121, Pluronic® R17R4, Pluronic® R25R4, Pluronic® R25R2, IGEPAL CA 630®, NONIDET NP-40, Nonidet® P40, Tween-20®, Tween-80®, Triton X-100™, Triton X-114™, Thesit®; the anionic detergent sodium dodecyl sulfate (SDS); the sugar stachyose; the condensing agent DMSO; and the chelator/DNAse inhibitor EDTA. Even more preferred are the auxiliary agents Nonidet® P40, Triton X-100™, Pluronic® F68, Pluronic® F77, Pluronic® F108, Pluronic® P65, Pluronic® P103, Pluronic® L31, Pluronic® L44, Pluronic® L61, Pluronic® L64, Pluronic® L92, Pluronic® R17R4, Pluronic® R25R4 and Pluronic® R25R2. Most preferred auxiliary agent is Pluronic® R25R2.

Optimal concentrations of auxiliary agents of the present invention are disclosed in U.S. Patent Application Publication No. 20020019358, which is incorporated herein by reference in its entirety. For example, in certain embodiments, pharmaceutical compositions of the present invention comprise about 5 ng to about 30 mg of a polynucleotide or a polynucleotide construct encoding IFNβ, or an active fragment or variant thereof, and about 0.001% (w/v) to about 2.0% (w/v) of Pluronic® R 25R4, preferably about 0.002% (w/v) to about 1.0% (w/v) of Pluronic® R 25R4, more preferably about 0.01% (w/v) to about 0.01% (w/v) of Pluronic® R 25R4, with about 0.01% (w/v) of Pluronic® R 25R4 being the most preferred; about 0.001% (w/v) to about 2.0% (w/v) of Pluronic® R 25R2, preferably about 0.001% (w/v) to about 1.0% (w/v) of Pluronic® R 25R2, more preferably about 0.001% (w/v) to about 0.1% (w/v) of Pluronic® R 25R2, with about 0.01% (w/v) of Pluronic® R 25R2 being the most preferred.

A pharmaceutical composition can be in solution form, or alternatively, in lyophilized form for reconstitution with a suitable vehicle, such as sterile, pyrogen-free water. Both liquid and lyophilized forms will comprise one or more agents, preferably buffers, in amounts necessary to suitably adjust the pH of the injected solution.

As defined herein, “treatment of a mammal” refers to the use of the method of the present invention to prevent, cure, retard, or reduce the severity of disease symptoms in a mammal; and/or result in no worsening in disease over a specified period of time. It is not required that the present invention totally cure or eliminate all disease symptoms.

The term “vertebrate” is intended to encompass a singular “vertebrate” as well as plural “vertebrates,” and comprises mammals and birds, as wells as fish, reptiles, and amphibians.

The term “mammal” is intended to encompass a singular “mammal” and plural “mammals,” and includes, but is not limited to humans; primate mammals such as apes, monkeys, orangutans, and chimpanzees; canine mammals such as dogs and wolves; feline mammals such as cats, lions, and tigers; equine mammals such as horses, donkeys, deer, zebra, and giraffe; and bears. Preferably, the mammal is a human subject.

The methods of present invention may be used to treat autoimmune diseases and other conditions related to inflammation in a mammal. Preferably, the methods of the present invention may be used to treat an autoimmune disease or an inflammatory demyelinating disease.

Multiple sclerosis (MS) is an inflammatory demyelinating disease of the central nervous system (CNS) that takes a relapsing-remitting or a progressive course. Its counterpart in the peripheral nervous system (PNS) is chronic inflammatory demyelinating polyradiculoneuropathy (CIDP). In addition, there are acute, monophasic disorders, such as the inflammatory demyelinating polyradiculoneuropathy termed Guillain-Barre syndrome (GBS) in the PNS, and acute disseminated encephalomyelitis (ADEM) in the CNS. Both MS and GBS are heterogeneous syndromes. In MS different exogenous assaults together with genetic factors can result in a disease course that finally fulfils the diagnostic criteria. In both diseases, axonal damage can add to a primarily demyelinating lesion and cause permanent neurological deficits. See Gold, R., et al., Mol. Med Today 6:88-91 (2000). Useful animal model exist which mimic certain features of human demyelinating diseases. Two models disclosed herein are experimental autoimmune encephalomyelitis (EAE) and neuritis (EAN) as models in rat and mouse strains.

Inflammatory demyelinating diseases to be treated by the methods of the present invention include, but are not limited to multiple sclerosis, Guillain-Barre Syndrome, experimental autoimmune encephalomyelitis and experimental autoimmune neuritis.

Examples of autoimmune diseases are multiple sclerosis; Sjogren's syndrome; sarcoidosis; insulin dependent diabetes mellitus; autoimmune thyroiditis; arthritis (e.g), osteoarthritis, rheumatoid arthritis, reactive arthritis, and psoriatic arthritis; ankylosing spondylitis; scleroderma; pernicious anemia (stomach), Addison's disease (adrenal glands), myasthenia gravis (acetylcholine receptors at neuromuscular junction), uveitis (eye), psoriasis (skin), Guillain-Barre Syndrome (nerve cells) and Grave's disease (thyroid). Systemic autoimmune diseases include systemic lupus erythematosus and dermatomyositis. Other diseases associated with inflammation include inflammation of the central nervous system (CNS) caused by fungal, bacterial and viral infection, inflammatory response to vaccination with live microorganisms, and local inflammation in response to trauma. Examples of fungal, bacterial and viral CNS infections include cerebral cryptococcosis, cryptococcal meningitis, cerebral malaria, pneumococcal meningitis, variant Creutzfeldt-Jakob disease, West Nile virus, poliomyelitis, paracoccidioidomycosis, neurocysticercosis, Epstein-Barr virus encephalitis, meningococcal meningitis, cerebral malaria, Venezuelan equine encephalomyelitis, St. Louis encephalitis, haemophilus influenzae meningitis, eastern equine encephalitis, streptococcal meningitis, nocardia meningitis, neurocysticercosis, neurosyphilis, toxoplasmosis, histoplasmosis and Japanese encephalitis. Also, the method of the present invention can be used to treat acute and chronic inflammatory disorders, to promote wound healing, and to prevent rejection after transplantation of cells, tissues, or organs.

Other examples of inflammatory conditions include asthma, eczema, atopical dermatitis, contact dermatitis, other eczematous dermatitides, seborrheic dermatitis, rhinitis, Lichen planus, Pemplugus, bullous Pemphigoid, Epidermolysis bullosa, uritcaris, angioedemas, vasculitides, erythemas, cutaneous eosinophilias, Alopecia areata, atherosclerosis, primary biliary cirrhosis and nephrotic syndrome. Related diseases include intestinal inflammations, such as Coeliac disease, proctitis, eosinophilia gastroenteritis, mastocytosis, inflammatory bowel disease, Chrohi's disease and ulcerative colitis, as well as food-related allergies.

Preferably, the methods of the present invention are used to treat multiple sclerosis, e.g., multiple sclerosis variants such as Neuromyelitis Optica (Decic's Disease), Diffuse Sclerosis, Transitional Sclerosis, Acute Disseminated Encephalomyelitis, and Optic Neuritis, but also Guillain-Barre's Syndrom, virus-, bacteria- or parasite-related demylinating or otherwise degenerative brain disease such as encphalopathies related to HIV, meningococcal or toxoplasma infections, central malaria, Lyme's disease etc.

Symptoms of MS which are prevented or ameliorated or treated include: weakness and/or numbness in one or more limbs; tingling of the extremities and tight band-like sensations around the trunk or limbs; dragging or poor control of one or both legs to spastic or ataxic paraparesis; hyperactive tendon reflexes; disappearance of abdominal reflexes; Lhermitte's sign; retrobulbar or optic neuritis; unsteadiness in walking; increased muscle fatiguability; brain stem symptoms (diplopia, vertigo, vomiting); disorders of micturition; hemiplegia; trigeminal neuralgia; other pain syndromes; nystagmus and ataxia; cerebellar-type ataxia; Charcot's triad; diplopia; bilateral internuclear ophthalmoplegia; myokymia or paralysis of facial muscles; deafness; tinnitus; unformed auditory hallucinations (because of involvement cochlear connections); vertigo and vomiting (vestibular connections); transient facial anesthesia or of trigeminal neuralgia; bladder dysfunction euphoria; depression; fatigue; dementia, dull, aching pain in the low back; sharp, burning, poorly localized pains in a limb or both legs and girdle pains; abrupt attacks of neurologic deficit; dysarthria and ataxia; paroxysmal pain and dysesthesia in a limb; flashing lights; paroxysmal itching; and/or tonic seizures, taking the form of flexion (dystonic) spasm of the hand, wrist, and elbow with extension of the lower limb.

The present invention is envisioned as retarding the onset of primary disease and/or relapse and reducing the severity of any or all symptoms of multiple sclerosis. The severity of the disease, and its subsequent relief, can be measured by a scale such as the Expanded Disability Status Scale (EDSS) described in Rudick and Goodkin, or a decrease in the frequency of relapses, or an increase in the time to sustained progression, or improvement in the magnetic resonance imaging (MRI) behavior in frequent, serial MRI studies.

For treatment of any of the above-described conditions, one or more polynucleotides, polynucleotide constructs, or compositions comprising such polynucleotides or constructs can be delivered locally, systemically or intra-cavity. In the “systemic delivery” embodiment of the invention, one or more polynucleotides, polynucleotide constructs, or compositions comprising a polynucleotide or polynucleotide construct, are administered into a tissue such that the IFNβ or an active variant or fragment thereof is expressed and the IFNβ polypeptide encoded by the polynucleotide or polynucleotide construct is released into the circulation, and such that a therapeutically effective amount of the IFNβ polypeptide is systemically delivered. Furthermore, polynucleotides or polynucleotide constructs encoding IFNβ or an active variant or fragment thereof may be delivered in combination with polynucleotides or polynucleotide constructs encoding other cytokines. Examples of combinations include polynucleotides or polynucleotide constructs encoding al IFNβ and IL-4; and polynucleotides or polynucleotide constructs encoding an IFNβ and TGFβ. Preferably, the polynucleotide, polynucleotide construct, or composition comprising a polynucleotide or polynucleotide construct, is administered free from ex vivo cells and free from ex vivo cellular material.

Administration can be into one or more tissues including but not limited to muscle, skin, brain, lung, liver, spleen, bone marrow, thymus, heart, e.g., myocardium, endocardium, and pericardium; lymph nodes, blood, bone, cartilage, pancreas, kidney, gall bladder, stomach, intestine, testis, ovary, uterus, rectum, nervous system, eye, gland, or connective tissue. Preferably, the administration is into muscle tissue, i.e., skeletal muscle, smooth muscle, or myocardium. Most preferably, the muscle is skeletal muscle. For polynucleotide constructs in which the polynucleotide or polynucleotide construct encoding an IFNβ is DNA, the DNA can be operably linked to a cell-specific promoter that directs substantial transcription of the DNA only in predetermined cells.

In certain embodiments, an IFNβ-encoding polynucleotide, polynucleotide construct, or composition comprising an IFNβ-encoding polynucleotide or polynucleotide construct, is delivered to any tissue including, but not limited to those disclosed herein, such that the polynucleotide or polynucleotide construct is free from association with liposomal formulations and charged lipids. Alternatively, the IFNβ-encoding polynucleotide, polynucleotide construct, or composition comprising an IFNβ-encoding polynucleotide or polynucleotide construct, is delivered to a tissue other than brain or nervous system tissue, for example, to muscle, skin, or blood, in any composition as described herein.

Furthermore, in the methods of the present invention, an IFNβ-encoding polynucleotide, polynucleotide construct, or composition comprising an IFNβ-encoding polynucleotide or polynucleotide construct may be administered to any internal cavity of a mammal, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, and the ocular cavities.

Preferably, an IFNβ-encoding polynucleotide, polynucleotide construct, or composition comprising an IFNβ-encoding polynucleotide or polynucleotide construct is delivered to the interstitial space of a tissue. “Interstitial space” comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

According to the disclosed methods, IFNβ-encoding polynucleotides, polynucleotide constructs, or compositions comprising an IFNβ-encoding polynucleotide or polynucleotide construct are preferably administered by intramuscular (i.m.), or subcutaneous (s.c.), routes. Other suitable routes of administration include intratracheal, transdermal, interdermal, intraocular, intranasal, inhalation, transmucosal (i.e., across a mucous membrane), intracavity (e.g., oral, vaginal, rectal, nasal, peritoneal, ventricular, or intestinal), and intravenous (i.v.) administration.

Any mode of administration can be used so long as the mode results in the expression of IFNβ or an active fragment or variant thereof, in the desired tissue, in an amount sufficient to be detectable, and/or prophylactically or therapeutically effective. Methods to detect polypeptides expressed in a mammal are well known to those of ordinary skill in the art and include, but are not limited to, serological methods to detect the polypeptide in serum, e.g., western blotting, staining tissue sections by immunohistochemical methods, measuring an immune response generated by the mammalian against the polypeptide, and measuring the activity of the polypeptide.

Administration means of the present invention include needle injection, catheter infusion, biolistic injectors, particle accelerators (e.g., “gene guns” or pneumatic “needleless” injectors) Med-E-Jet (Vahlsing, H., et al., J. Immunol. Methods 171,11-22 (1994)), Pigjet (Schrijver, R., et al., Vaccine 15, 1908-1916 (1997)), Biojector (Davis, 14., et al., Vaccine 12,1503-1509 (1994); Gramzinski, R., et al., Mol Med 4,109-118 (1998)), AdvantaJet (Linmayer, I., et al., Diabetes Care 9:294-297 (1986)), Medi-jector (Martins, J., and Roedl, E. J. Occup. Med. 21:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65, 2193-2203 (1999)) or topical applications during surgery. The preferred modes of administration are intramuscular needle-based injection and pulmonary application via catheter infusion. Each of the references cited in this paragraph is incorporated herein by reference in its entirety.

Determining an effective amount of a composition depends upon a number of factors including, for example, the chemical structure and biological activity of the substance, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian.

In one embodiment, an IFNβ-encoding polynucleotide, polynucleotide construct, or composition comprising an IFNβ-encoding polynucleotide or polynucleotide construct is administered free from association with liposomal formulations, charged lipids, or transfection-facilitating viral particles. In another embodiment, an IFNβ-encoding polynucleotide, polynucleotide construct, or composition comprising an IFNβ-encoding polynucleotide or polynucleotide construct is administered free from association with any delivery vehicle now known in the art that can facilitate entry into cells.

As used herein, “ex vivo” cells are cells into which the polynucleotide construct is introduced, for example, by transfection, lipofection, electroporation, bombardment, or microinjection. The cells containing the polynucleotide construct are then administered in vivo into mammalian tissue. Such ex vivo polynucleotide constructs are well-known to those of ordinary skill in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J. -F. et al., Cancer Gene Therapy 3: 31-38 (1996).

In the “local delivery” embodiment of the present invention, an IFNβ-encoding polynucleotide, polynucleotide construct, or composition comprising an IFNβ-encoding polynucleotide or polynucleotide construct is administered in vivo at or near a disease site (e.g., site of inflammation), such that the polynucleotide is incorporated into the local cells at the site of inflammation. The local cells subsequently express the IFNβ polypeptide or an active fragment or variant thereof in an amount effective to treat the inflammatory condition.

In this embodiment, an IFNβ-encoding polynucleotide, polynucleotide construct, or composition comprising an IFNβ-encoding polynucleotide or polynucleotide construct can be administered into a site of inflammation. Alternatively, an IFNβ-encoding polynucleotide, polynucleotide construct, or composition comprising an IFNβ-encoding polynucleotide or polynucleotide construct can be administered into cells surrounding a site of inflammation, near a site of inflammation, or adjacent to a site of inflammation, such that a therapeutically effective amount of the cytokine is produced in vivo near or within the site of inflammation. One way to provide local delivery of an IFNβ-encoding polynucleotide, polynucleotide construct, or composition comprising an IFNβ-encoding polynucleotide or polynucleotide construct is by administering intravenously a polynucleotide construct comprising a tissue-specific targeted promoter, wherein the polynucleotide is incorporated into the cells affected by inflammation and the cytokine is expressed in an amount effective to treat the inflammatory condition. The local delivery embodiment is preferred for arthritis or conditions related to cell, tissue, or organ transplantation.

An IFNβ-encoding polynucleotide, polynucleotide construct, or composition comprising an IFNβ-encoding polynucleotide or polynucleotide construct can be administered either within ex vivo cells or free of ex vivo cells or ex vivo cellular material. Preferably, the polynucleotide construct is administered free of ex vivo cells or ex vivo cellular material.

An IFNβ-encoding polynucleotide, polynucleotide construct, or composition comprising an IFNβ-encoding polynucleotide or polynucleotide construct to be delivered can be solubilized in a buffer prior to administration. Suitable buffers include, for example, phosphate buffered saline (PBS), normal saline, Tris buffer, and sodium phosphate vehicle (100-150 mM preferred). Insoluble polynucleotides can be solubilized in a weak acid or base, and then diluted to the desired volume with a neutral buffer such as PBS. The pH of the buffer is suitably adjusted, and moreover, a pharmaceutically acceptable additive can be used in the buffer to provide an appropriate osmolarity within the lipid vesicle. Preferred salt solutions and auxiliary agents are disclosed herein.

A systemic delivery embodiment can be particularly useful for treating nonlocalized disease conditions (i.e., multiple sclerosis), or a disease category that might be responsive to continuous exposure by the systemic route. A local delivery embodiment can be particularly useful for treating disease conditions that might be responsive to high local concentration i.e. transplantation related conditions. When advantageous, systemic and local delivery can be combined.

U.S. Pat. Nos. 5,589,466, 5,693,622, 5,580,859, 5,703,055, and PCT international patent application PCT/US94/06069 (WO 94/29469), the disclosures of which are incorporated herein by reference in their entireties, provide methods for delivering compositions comprising naked DNA, or DNA cationic lipid complexes to mammals.

Compositions used in of the present invention can be formulated according to known methods. Suitable preparation methods are described, for example, in Remington's Pharmaceutical Sciences, 16^{th} Edition, A. Osol, ed., Mack Publishing Co., Easton, Pa. (1980), and Remington's Pharmaceutical Sciences, 19^{th} Edition, A. R. Gennaro, ed., Mack Publishing Co., Easton, Pa. (1995), both of which are incorporated herein by reference in their entireties. Although the composition is preferably administered as an aqueous solution, it can be formulated as an emulsion, gel, solution, suspension, lyophilized form, or any other form known in the art. According to the present invention, if the composition is formulated other than as an aqueous solution, it will require resuspension in an aqueous solution prior to administration. In addition, the composition may contain pharmaceutically acceptable additives including, for example, diluents, binders, stabilizers, and preservatives.

For aqueous compositions used in vivo, the use of sterile pyrogen-free water is preferred. Such formulations will contain an effective amount of a polynucleotide or polynucleotide construct together with a suitable amount of an aqueous solution in order to prepare pharmaceutically acceptable compositions suitable for administration to a mammal.

The present invention also provides kits for use in treating inflammatory autoimmune diseases and other conditions related to inflammation comprising an administration means and a container means containing one or more IFNβ-encoding polynucleotide or polynucleotide constructs in a sterile environment. Preferably, the polynucleotide or polynucleotide construct is in the amount of 1 ng to 30 mg, more preferably in the amount of 100 ng to 20 mg.

The cytokine encoded by the polynucleotide or polynucleotide construct of the kit of the present invention can be an IFNβ and one or more additional cytokines, including any of the cytokines described herein. The construct can be in the form of a pharmaceutical composition and can contain a pharmaceutically acceptable carrier. Pharmaceutical compositions are described above. The kit can further comprise a pharmaceutically acceptable carrier in a separate container means.

Any suitable container or containers can be used with pharmaceutical kits. Examples of containers include, but are not limited to, glass containers, plastic containers, or strips of plastic or paper.

The container in which the composition is packaged prior to use can comprise a hermetically sealed container enclosing an amount of the lyophilized formulation or a solution containing the formulation suitable for a pharmaceutically effective dose thereof, or multiples of an effective dose. The composition is packaged in a sterile container, and the hermetically sealed container is designed to preserve sterility of the pharmaceutical formulation until use. Optionally, the container can be associated with administration means and/or instruction for use.

Administration means include, but are not limited to syringes and needles, catheters, biolisitic injectors, particle accelerators, i.e., “gene guns,” pneumatic “needleless” injectors, gelfoam sponge depots, other commercially available depot materials, e.g., hydrogels, osmotic pumps, and decanting, polynucleotide coated sutures, skin patches, or topical applications during surgery. In one embodiment, the administrative means is a syringe with a plunger associated with the syringe. In another embodiment, the container is a syringe and the administration means is a plunger.

The kit can further comprise an instruction sheet for administration of the composition to a mammal. The polynucleotide components of the composition are preferably provided as a liquid solution or they may be provided in lyophilized form as a dried powder or cake. If the polynucleotide or polynucleotide construct is provided in lyophilized form, the dried powder or cake may also include any salts, auxiliary agents, trnasfection facilitating agents, and additives of the composition in dried form. Such a kit may further comprise a container with an exact amount of sterile pyrogen-free water, or any buffer described herein, including PBS, normal saline, Tris buffer, and sodium phosphate vehicle, for precise reconstitution of the lyophilized components of the composition.

Having now generally described the invention, the same will become more readily understood by reference to the following specific examples which are included herein for purposes of illustration only and are not intended to be limiting unless otherwise specified.

### EXAMPLES

### Example 1

### Construction and Evaluation of Murine IFNβ Expression Vectors

The complete coding region of the murine beta-interferon gene (nucleotides 1270 to 1818 of GenBank accession number X14029 (SEQ ID NO:3), having the amino acid sequence of GenBank accession number CAA32190 (SEQ ID NO:4)) was PCR amplified from genomic DNA.

SEQ ID NO:3

| | |
|---|---|
| 1270 | a tgaacaacag gtggatcctc cacgctgcgt tcctgctgtg |

cttctccacc

| | |
|---|---|
| 1321 | acagccctct ccatcaacta taagcagctc cagctccaag |

aaaggacgaa cattcggaaa

| | |
|---|---|
| 1381 | tgtcaggagc tcctggagca gctgaatgga aagatcaacc |

tcacctacag ggcggacttc

| | |
|---|---|
| 1441 | aagatcccta tggagatgac ggagaagatg cagaagagtt |

acactgcctt tgccatccaa

| | |
|---|---|
| 1501 | gagatgctcc agaatgtctt tcttgtcttc agaaacaatt |

tctccagcac tgggtggaat

| | |
|---|---|
| 1561 | gagactattg ttgtacgtct cctggatgaa ctccaccagc |

agacagtgtt tctgaagaca

| | |
|---|---|
| 1621 | gtactagagg aaaagcaaga ggaaagattg acgtgggaga |

tgtcctcaac tgctctccac

| | |
|---|---|
| 1681 | ttgaagagct attactggag ggtgcaaagg taccttaaac |

tcatgaagta caacagctac

| | |
|---|---|
| 1741 | gcctggatgg tggtccgagc agagatcttc aggaactttc |

tcatcattcg aagacttacc

| | |
|---|---|
| 1801 | agaaacttcc aaaactga |

SEQ ID NO:4

| | |
|---|---|
| 1 | mnnrwilhaa fllcfsttal sinykglglg ertnirkcge |

lleglngkin ltyradfkip

| | |
|---|---|
| 61 | memtekmgks ytafaigeml gnvflvfrnn fsstgwneti |

vvrlldelhg gtvflktvle

| | |
|---|---|
| 121 | ekgeerltwe msstalhlks yywrvgrylk lmkynsyawm |

| | |
|---|---|
| vvraeifrnf liirrltrnf | 181 |

gn

The coding sequence was PCR amplified with Pfu polymerase (Stratagene, San Diego, Calif.) using the following two primers (Genosys, Woodlands, Tex.) according to the manufacturer's recommendation:

Forward primer: 5′ GCG GAA TTC GCC GCC ACC ATG AAC AAC AGG TGG ATC CTC 3′ (SEQ ID NO:5); and

Reverse primer: 5′ GCG GCT AGC TCA GTT TTG GAA GTT TCT GGT A 3′ (SEQ ID NO:6).

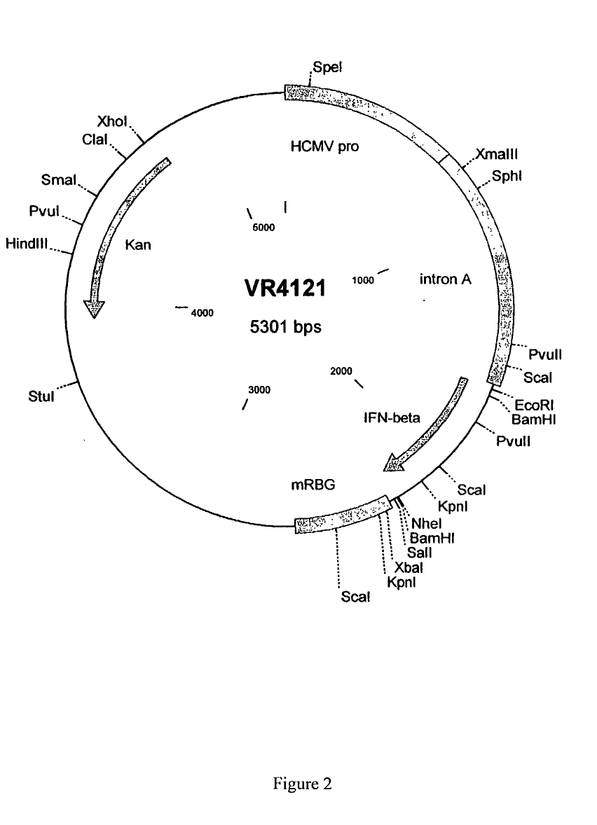

The forward primer includes the consensus Kozak translational initiation signal GCC GCC ACC (SEQ ID NO:7) at the 5′ end. One microgram of mouse genomic DNA was amplified by PCR using the following conditions: 1 cycle of 95° C. for 5 minutes, followed by 35 cycles of 95° C. 30 seconds, 55° C. for 30 seconds, and 72° C. for 1 minute, followed by 1 cycle of 72° C. for 5 minutes. The 569 base pair PCR product was digested with EcoRI plus NheI and ligated into the same sites of the expression vector VR1051. Other expression vectors, e.g. VR1055, can also be used. VR1051 and VR1055 contain a kanamycin-resistance gene, a human cytomegalovirus (CMV) immediate early gene I promoter/enhancer, 5′ untranslated sequence, CMV intron A sequence and a transcriptional terminator region derived from the rabbit β-globin gene (Hartikka, J., et al., Hum. Gene Ther. 7:1205-1217 (1996)). The resulting construct is designated VR4121 (FIG. 2), the sequence of which is depicted herein as SEQ ID NO:8.

pDNA was prepared by bacterial fermentation (Hartikka, J., et al., Hum. Gene Ther. 7:1205-1217 (1996)) and purified by double cesium chloride/ethidium bromide ultracentrifugation followed by ethanol precipitation and dialysis. pDNA used in these studies was free of detectable RNA, and endotoxin levels were less than 0.06 endotoxin units/μg pDNA.

VM92 murine melanoma cells were plated in 6-well plates at a concentration of 2×10⁵ cells per well. One day later, the medium was removed and the cells were washed with PBS followed by addition of either mIFNβ pDNA (VR4121) or control pDNA (e.g., VR1055, backbone plasmid lacking the IFNβ gene) and DMRIE/DOPE complex (1:1, 1 μg of each, 1 ml/well) in Optimem medium (Life Technologies/Invitrogen, San Diego, Calif.). Alternative control plasmids, e.g. VR1051, can also be used. After 4-5 hrs, 1 ml Optimem with 30% fetal calf serum (FCS) was added. One day later, 1 ml Optimem with 10% FCS was added. The supernatants were collected 24 hrs after the start of the in vitro transfection.

The supernatants from cells in vitro transfected with either mIFNβ pDNA (VR4121) or control pDNA (VR1055) were assayed for antiviral activity against murine encephalomyocarditis virus (EMCV) infection of murine L929 cells (IIT Research Institute, Chicago, Ill.). Briefly, L929 cells were aliquoted into 96-well plates (3×10⁴ cells/well) and incubated for 24 hrs. The culture medium was then removed and serial dilutions of supernatants from the in vitro transfections were added to the wells. Both a murine IFN α/β reference standard and tissue culture medium were included as controls. After 24 hrs incubation, the wells were washed and EMCV was added at a multiplicity of infection of 0.02. After a 24 hr incubation, the wells were washed, fixed with 5% formalin and stained with 1% crystal violet. Samples with IFN activity protected the cells and resulted in darkly stained monolayers. The lowest dilution having IFN activity was used to calculate the IFN titer relative to the IFN protein standard.

The supernatants from cells in vitro transfected with either mIFNβ pDNA (VR4121) or control pDNA (VR1055) were assayed for antiproliferative activity against murine melanoma B16F10 cells using the Boehringer Mannheim Cell Proliferation Kit II (XTT) (Roche Molecular Biochemicals, Indianapolis, Ind.). Alternative control plasmids, e.g. VR1051, can also be used. B16F10 cells were aliquoted into 96-well plates (10³ cells/well) and incubated for 24 hrs. The culture medium was then removed and the supernatants from the in vitro transfections were added to the wells. Both murine IFN α protein (ICN, Costa Mesa, Calif.) and tissue culture medium were included as controls. After 48 hrs incubation, the XTT labeling reagent was added to the wells and allowed to incubate for 6 hrs. The plates were read at 490-690 nm and the percent reduction in cell proliferation was calculated using the following formula:

$$1 - \frac{{OD}_{490 - 690} of cells incubated with mIFN β pDNA supernatants}{{OD}_{490 - 690} of cells incubated with control pDNA supernatants} \times 100$$

I- OD₉₀₆₉₀ of cells incubated with mIFNfpi,DNA supematants x 100 OD₄₉₀₆₉₀ of cells incubated with control pDNA supennatants

Supernatants from cells in vitro transfected with IFNβ pDNA (VR4121) were evaluated in in vitro assays of IFN α/β antiviral activity and cell proliferation activity. The IFNβ pDNA supernatants had 30,000 Units/ml of IFN α/β antiviral activity and resulted in a 53% reduction in murine melanoma cell proliferation. Supernatants from control pDNA had no detectable IFN activity in either assay.

### Example 2

### Intramuscular Injection of IFNβ Expression Vectors in EAE Mice

EAE was induced in 5-week old SJL/J mice by subcutaneous (s.c.) injection of an emulsion of myelin basic protein (MBP) (Sigma, St Louis, Mo.) and H37RA Mycobacterium tuberculosis (M.Tb.) in Freund's incomplete adjuvant (Difco/Becton Dickinson, Franklin Lakes, N.J.). Other mouse strains, e.g. C3H mice, may also be used. The emulsion was formed by mixing the MBP, M. Tb. and Freund's incomplete adjuvant in a tissue homogenizer (Fisher Scientific, Pittsburgh, Pa.). Each mouse received 400 μg of MBP and 200 μg of M. Tb in a total volume of 100 μl on days 0 and 7. The emulsion was injected s.c. at the base of the tail and on the flank (100 μl per site) on anesthetized mice, using a 1 cc syringe fitted with a 22 g needle.

For the pDNA therapy, mice were i.m. injected with 100 μg IFNβ pDNA (VR4121) or control pDNA (VR1055) on days −2 and +5, relative to the first MBP and M. Tb. injection (n=15 mice per group). Alternative control plasmids, e.g. VR1051, can also be used. Mice were marked with a numbered ear tag and a master list was prepared of each mouse and the treatment received. After the final pDNA injection, mice were randomized and scored in a blinded fashion by two investigators daily up to day 18 and then three times per week thereafter. The neurological symptoms were scored using the following key: 0=no symptoms, 1=tail weakness, 2=flaccid tail, 3=impaired righting reflex, 4=partial hind limb paralysis, 5=complete hind limb paralysis, 6=moribund.

The i.m. injections were performed using a sterile 300 μl tuberculin syringe attached to 28G ½ needle (Becton Dickinson, Franklin Lakes, N.J.). A plastic collar from a 200 μl pipette tip was attached to the needle to prevent it from penetrating beyond 2 mm into the muscle. Mice were injected i.m. with 100 μg pDNA in 100 μl of 150 mM sodium phosphate, pH 7.2. The injection was split between the two rectus femoris muscles of each rear hind leg (50 μg/50 μl per muscle).

The Mann-Whitney U nonparametric statistical test was used to identify significant differences between treatment groups. Differences were considered statistically significant when the P value was <0.05.

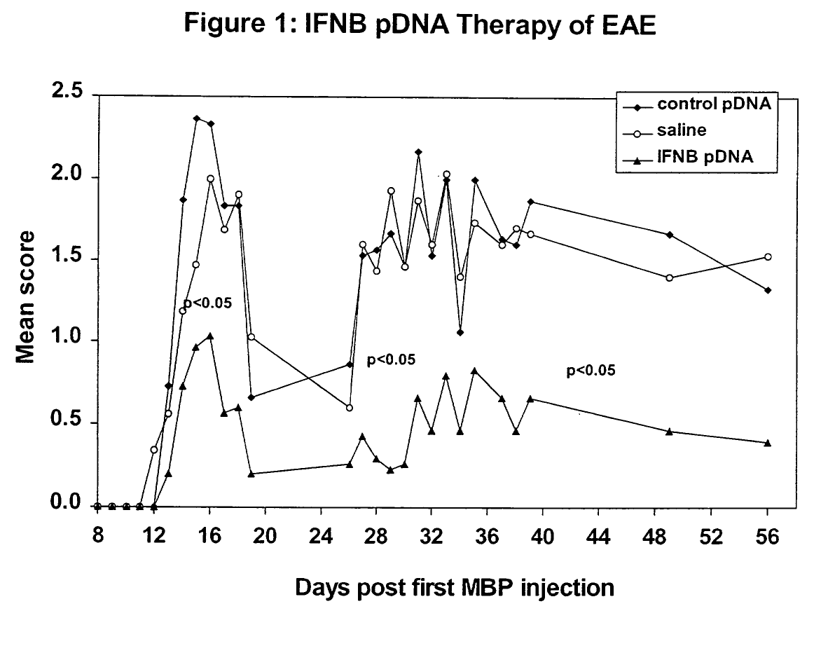

Mice receiving 100 μg IFNβ pDNA on days −2 and +5 had a significant reduction in neurological symptoms during the primary disease attack (p<0.05) (FIG. 1). Furthermore, mice treated with IFNβ had a significant reduction in symptoms during the relapse phase of disease and this effect continued up to day 60 (p<0.05). There was no significant difference between mice treated with control pDNA versus saline.

The results of these studies demonstrate that delivery of IFNβ by i.m. injection of pDNA encoding IFNβ can have a significant therapeutic effect in a mouse model of multiple sclerosis. Only two injections were required to elicit a significant therapeutic effect. This is in marked contrast to every other day or weekly injections required for IFNβ protein therapy. One explanation for the long-term effect observed after i.m. injection of IFNβ pDNA is that muscle cells have become transfected in vivo with IFNβ pDNA and continue to express the IFNβ gene product over many weeks. Hence, pDNA delivery of IFNβ may allow for less frequent administration of IFNβ, compared to protein therapy.

Although the IFNβ gene was delivered in a previous study via intracerebral (i.c.) injection of a pDNA/lipid complex, attempts to deliver pDNAs encoding cytokines other than IFNβ (IFNβ pDNA was not used) by i.m. injection of naked DNA were unsuccessful (Croxford, J. L., et al., J. Immunol. 160:5181-5187 (1998)), possibly due to problems with the plasmid vector used. In the present research, we demonstrated that i.m. injection of IFNβ-encoding pDNA can significantly reduce the clinical score in the EAE model. By requiring less frequent injections than with the delivery of IFNβ protein, the therapy should be easier for patients to use and may reduce the incidence of irritation at the injection site. In addition, some MS patients have experienced flu-like symptoms after injection of IFNβ protein (Lublin, F. D., et al, Neurology 46:12-18 (1996)). The side effects of IFNβ protein therapy may be related to the high serum levels occurring in the first 8-48 hrs after injection of the protein (Chiang, J., et al., Pharmaceutical Research 10:567-572 (1993); Alam, J., et al, Pharmaceutical Research 14:546-549 (1997)). In contrast, i.m. injection of IFNβ pDNA may result in lower, more stable, serum levels leading to fewer side effects.

The findings of this research are important for development of a pDNA-based therapy for MS requiring much less frequent injections. The fact that the IFN, pDNA therapy reduced clinical scores in both the primary disease and in the relapse phase in the EAE model, e.g. Examples 3, 4, 6, and 7, illustrates the long-term efficacy of this therapy.

### Example 3

### Intramuscular IFNβ pDNA Therapy of Primary EAE Post Disease Induction

EAE is induced in 4-week old female SJL/J mice (Jackson) as follows. On day 0, mice are anesthetized with metophane, ear tagged and injected subcutaneously with 400 μg MBP and Freund's incomplete adjuvant with 400 μg Mycobacterium tuberculosis. The MBP/M. tuberculosis emulsion is made as follows: 400 fig MBP is diluted in 50 μl saline, 400 μg M. tuberculosis diluted in 50 μl of Freund's Incomplete Adjuvant. The 2 solutions are mixed with tissue homogenizer until a stable emulsion is formed. After anesthetizing the mice with metophane, each mouse is injected with 100 μl of the emulsion at 2 sites (50 μl in the flank and 50 μl near the base of the tail) on day 0 and again on day 7 with a 1 cc syringe fitted with a 22 g needle. On day 2,2 treatment groups are created having equal weights per group and a master list of treatment/mouse is created.

On day 2 and 5, mice are given a bilateral im injection in the rectus femoris of 100 μg VR1051 or V1055 (control) or VR4121 (mIFNβ) in 150 mM sodium phosphate. On day 10, mice are randomized to allow for scoring in a blind fashion and the mice are scored daily for neurological symptoms and weight. On day 16, the study is unblinded.

The EAE scoring guide is shown in Table 1.

TABLE 1
EAE Scoring Guide

| Score | Signs |
|---|---|
| 1 | tail weakness (tail can be held up briefly but then flops down) |
| 2 | tail limpness (tail can not be held up) |
| 3 | partially impaired righting reflex (takes 1-3 seconds to right) |
| 4 | partial hind limb paralysis (one foot dragging) or grossly impaired |
| | righting reflex (takes 4-10 seconds to right or cannot right) |
| 5 | complete hind limb paralysis (both legs dragging) |
| 6 | moribund |

### Example 4

### Intramuscular IFNβ pDNA Therapy of Relapsing EAE

EAE is induced in mice on day 0 as described in Example 3. On day 10, the mice are monitored daily by determining neurological score and weight.

On day 19, mice are selected for each treatment group based on neurological scores in the primary attack. Each treatment group contains mice with an equivalent range of scores such that graphs of average score per group overlap. Weights are checked and treatment groups contain mice of equivalent weights. A master list of treatment/mouse is created. On days 19 and 22, 26, and 29, mice are given a bilateral i.m. injection in the rectus femoris of 100 μg VR1051 or V 1055 (control) or 100 μg VR4121 (IFNβ) in 150 mM sodium phosphate. Mice continue to be scored and weighed during this time period 3 times per week. On day 35, the study is unblinded. Mice are scored according to the EAE scoring guide, shown in Table 1.

### Example 5

### IFNβ pDNA Therapy for MS in Human

The complete coding region of the human beta-interferon gene (nucleotides 1 to 564 of GenBank accession number NM_{—}002176 (SEQ ID NO:1), having the amino acid sequence of GenBank accession number NP_{—}002167 (SEQ ID NO:2)) was PCR amplified from human genomic DNA.

SEQ ID NO:1

| | | |
|---|---|---|
| 1 | atgaccaaca agtgtctcct ccaaattgct ctcctgttgt gcttctccac tacagctctt | |
| 61 | tccatgagct acaacttgct tggattccta caaagaagca gcaattttca gtgtcagaag | |
| 121 | ctcctgtggc aattgaatgg gaggcttgaa tattgcctca aggacaggat gaactttgac | 181 |
| | atccctgagg agattaagca gctgcagcag ttccagaagg aggacgccgc attgaccatc | 241 |
| | tatgagatgc tccagaacat ctttgctatt ttcagacaag attcatctag cactggctgg | 301 |
| | aatgagacta ttgttgagaa cctcctggct aatgtctatc atcagataaa ccatctgaag | 361 |
| | acagtcctgg aagaaaaact ggagaaagaa gattttacca ggggaaaact catgagcagt | 421 |
| | ctgcacctga aaagatatta tgggaggatt ctgcattacc tgaaggccaa ggagtacagt | 481 |
| | cactgtgcct ggaccatagt cagagtggaa atcctaagga acttttactt cattaacaga | 541 |
| | cttacaggtt acctccgaaa ctga | |

SEQ ID NO:2

| | |
|---|---|
| −21 mtnkcllqia lllcfsttal smsynhlgfl qrssnfqcqk llwqlngrle yclkdrmnfd | 40 |
| ipeeikqlqq fqkedaalti yemlqnifai frqdssstgw netivenlla nvyhqinhlk | |
| 100 tvleekleke dftrgklmss lhlkryygri ihylkakeys hcawtivrve ilrnfyfinr | 160 |
| ltgylrn | |

The coding sequence was PCR amplified with Taq polymerase (Stratagene, San Diego, Calif.) using the following two primers (Genosys, Woodlands, Tex.) according to the manufacturer's recommendation:

Forward primer: 5′ GCG GAA TTC GCC GCC ACC ATG ACC AAC AAG TGT CTC CTC 3′ (SEQ ID NO:9); and

Reverse primer: 5′ GCG GCT AGC TCA GTT TCG GAG GTA ACC TG 3′ (SEQ ID NO:10).

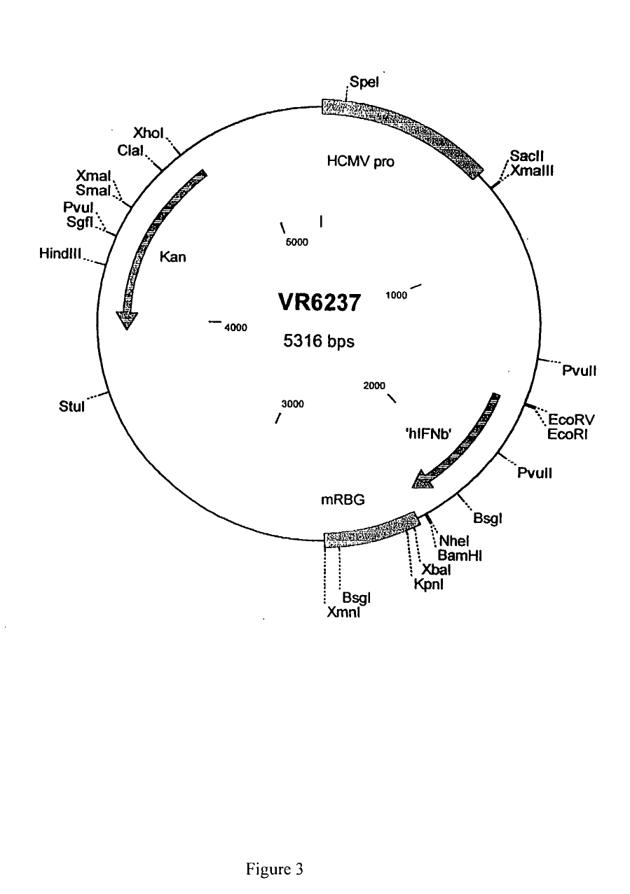

The PCR product was digested with EcoRI plus NheI and ligated into the same sites of the expression vector VR1051. Alternative vectors, e.g. VR1055, may also be used. VR1051 and VR1055 contain a kanamycin-resistance gene, a human cytomegalovirus (CMV) immediate early gene I promoter/enhancer, 5′ untranslated sequence, CMV intron A sequence and a transcriptional terminator region derived from the rabbit β-globin gene (Hartikka, J., et al., Hum. Gene Ther. 7:1205-1217 (1996)). The resulting construct is designated VR6237 (FIG. 3), the sequence of which is depicted herein as SEQ ID NO:11.

Verification of biological activity of VR6237 was tested by the following method. Murine melanoma VM92 cells were plated at a concentration of 2×10⁵ cells per well in a 6 well plate and incubated for 24 h. Medium was removed from the cells which were washed with PBS followed by addition of VR6237 (hIFN) pDNA and cationic lipid DMRIE/DOPE complex (1:1, 1 mg of each, 1 ml/well) in Optimem medium (Life Technologies, Gaithersburg, Md.). After 4-5 h incubation at 37° C., one ml of Optimem with 30% fetal calf serum (FCS) was added to each well, followed by addition of one ml of Optimem with 10% FCS the next day. The supernatants from the VR6237 in vitro transfections were collected 48 h after the start of the transfection.

The tissue culture supernatants were analyzed in an in vitro anti-viral assay (IIT Institute, Chicago, Ill.) according to the following protocol. Human A549 cells were plated into 96-well plates at 2.5×10⁴ cells/well. After 24 hrs incubation, the culture medium was removed and serial dilutions of the test samples were added to duplicate wells. A hIFNα reference standard (NIH) was included as a positive control and cells incubated with tissue culture medium alone were included as negative controls. After a 24 hr incubation period, the samples were removed, the cells washed twice, and 100 ml of murine encephalomyocarditis virus was added to the wells (500 TCID₅₀ units per well). After a 24 hr incubation period, the culture medium was removed, the cells were washed, fixed with 5% formalin, and stained with 1% crystal violet. Samples with IFN activity protected the cells from virus infection, resulting in darkly stained monolayers. The lowest dilution having IFN activity was used to calculate the IFN titer relative to the IFN standard.

The in vitro anti-viral assay was also run using murine L929 cells following the same protocol as for the human A549 cells except murine IFNα/β (NIH) was used as the reference standard. The results are shown in Table 2.

TABLE 2

| Cell Line | Activity (U/ml) |
|---|---|
| Human A549 | 30,000 |
| Murine L929 | 3000 |

pDNA was prepared by bacterial fermentation (Hartikka, J., et al., Hum. Gene Ther. 7:1205-1217 (1996)) and purified by double cesium chloride/ethidium bromide ultracentrifugation followed by ethanol precipitation and dialysis. pDNA used in these studies is free of detectable RNA, and endotoxin levels are less than 0.06 endotoxin units/ug pDNA.

To treat MS (e.g., primary progressive, secondary progressive, or relapsing-remitting) in human patients, 10 ng-30 mg, preferably 200 μg-20 mg, preferably 1-10 mg of IFNβ-encoding plasmid DNA (e.g., VR6237) in a pharmacologically acceptable carrier is delivered to patients one to three times during the first week of treatment and intermittently thereafter (e.g., biweekly or monthly) by injection, preferably intramuscularly. The therapy regimen is continued indefinitely, or at least for three months to one year, during which time the patients are monitored for (a) Kurtzke Expanded Disability Status Scale (EDSS) score changes from baseline (Kurtzke, J. F., Neurology 33:1444-1452 (1983); (b) annual exacerbation rates; and (c) the severity of exacerbation based in the Scripps Neurologic Rating Scale (NRS; Sipe, J. C., et al., Neurology 34:1368-1372 (1984). Patients are also monitored by annual MRI to determine the change in lesion area from baseline.

### Example 6

### IFNβ pDNA Therapy of Primary Disease in the EAE Model

EAE was induced in 6-week old SJL/J mice by s.c. injection of an emulsion of proteolipid protein (PLP₁₃₉₋₁₅₁, HCLGKWLGHPDKF) (SEQ ID NO:12) (Biosynthesis Inc., Lewisville, Tex.) and Mycobacterium tuberculosis (M Tb.) in Freund's incomplete adjuvant (Difco/Becton Dickinson, Franklin Lakes, N.J.). The emulsion was made as follows: 40 μg PLP was diluted in 75 μl phosphate buffered saline (PBS, Sigma) and 75 μg of M Tb. was diluted in 75 μl of Freund's incomplete adjuvant. The 2 solutions were mixed with a tissue homogenizer until a stable emulsion was formed. After anesthetizing the mice with metophane, the mice were injected with 150 μl of the emulsion at 4 sites (37.5 μl per site, into the base of the tail on each side and high on the flank on each side). The PLP/M. Tb emulsion was injected on day 0 with a 1 cc syringe fitted with a 22 g needle.

For pDNA therapy of the primary attack in the PLP model, mice were i.m. injected with 100 μg of IFNβ-encoding pDNA (VR4121) or control pDNA (VR1055) on days 2 and 5, relative to the PLP injection (n=14 mice per group). The i.m. injections were performed using a sterile 300 μl tuberculin syringe attached to a 28G ½ needle (Becton Dickinson, Franklin Lakes, N.J.). A plastic collar from a 200 μl pipette tip was attached to the needle to prevent it from penetrating beyond 2 mm into the muscle. Mice were injected i.m. with 100 μg pDNA in 100 μl of 150 mM sodium phosphate, pH 7.2 to which 0.01% Pluronic®-R 25R2 (described in U.S. Patent Application No. 2002/0019358 A1, published Feb. 14, 2002, and which is incorporated herein by reference in its entirety) was added immediately prior to injection. The injection was split between the two rectus femoris muscles of each hind leg (50 μg/50 μl per muscle).

Mice were marked with a numbered ear tag and a master list was prepared of each mouse and the treatment received. After the final pDNA injection, mice were randomized and scored in a blinded fashion by two investigators 3× per week. The neurological symptoms were scored using the following key: 0=no symptoms, 1=tail weakness, 2=flaccid tail, 3=partially impaired righting reflex, 4=partial hind limb paralysis or grossly impaired righting reflex, 5=complete hind limb paralysis, 6=moribund. Mice were followed out to day 26 at which time the study was unblinded.

The Mann Whitney U non-parametric test was used to identify significant differences between treatment groups. Differences were considered statistically significant when the P value was <0.05.

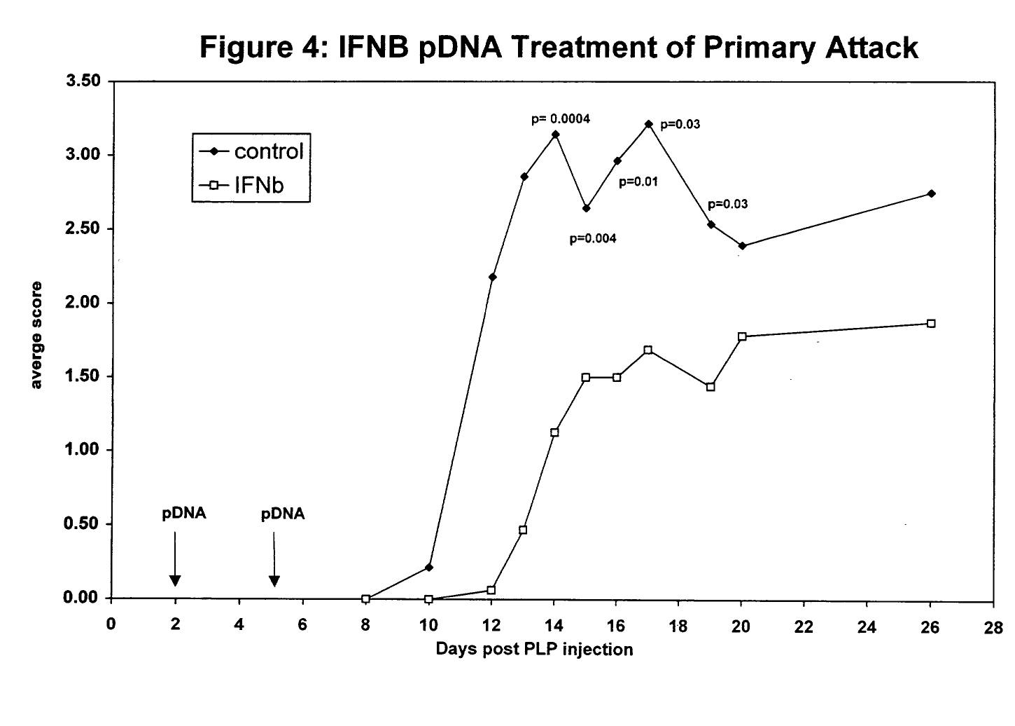

Mice receiving 100 μg IFNβ-encoding pDNA on days 2 and 5 had a significant reduction in neurological symptoms during the primary attack of disease (p<0.05) (FIG. 4). The results of these studies demonstrate that delivery of IFNβby i.m. injection of pDNA encoding IFNβ can have a significant therapeutic effect in a PLP-induced model of multiple sclerosis. This model permits the evaluation of treatment of disease that presents with symptoms of greater severity than the MBP-induced model of disease. As shown in the treatment of MBP-induced EAE, only two injections were required to elicit a significant therapeutic effect on the primary phase of the disease, demonstrating that pDNA delivery of IFNβ allows for less frequent administration of IFNβ, compared to protein therapy.

### Example 7

### IFNβ pDNA Therapy of the Relapse Phase of EAE

EAE was induced in 6-week old SJL/J mice by s.c. injection of an emulsion of proteolipid protein (PLP₁₃₉₋₁₅₁, HSLGKWLGHPDKF) (SEQ ID NO: 13) (Biosynthesis Inc., Lewisville, Tex.) and Mycobacterium tuberculosis (M Tb.) in Freund's incomplete adjuvant (Difco/Becton Dickinson, Franklin Lakes, N.J.). The emulsion was made as described in Example 6. After anesthetizing the mice with isoflurane (Henry Schein, Melville, N.Y.), the mice were injected with 150 μl of the emulsion at 4 sites (37.5 μl per site, into the base of the tail on each side and high on the flank on each side). The PLP/M Tb emulsion was injected on day 0 with a 1 cc syringe fitted with a 22 g needle.

For therapy of the relapse phase of the disease, mice were marked with a numbered ear tag and scored by two investigators 3× per week. The neurological symptoms were scored using the key described in Example 6. On day 18 after PLP/M. Tb injection, mice having scores that reached 2 or greater during the primary attack (days 11-18) were randomized into two groups such that each group had a similar profile of scores during the primary attack (n=9 mice per group). One group was i.m. injected with 100 μg VR1055 (control pDNA) and the other group was i.m. injected with 100 μg VR4121 (mIFNβ-encoding pDNA) in 100 μl of 150 mM sodium phosphate, pH 7.2 to which 0.01% Pluronic®-R 25R2 was added immediately prior to injection. The injection was split between the two rectus femoris muscles of each hind leg (50 μg/50 μl per muscle). Mice were injected i.m. with pDNA twice per week for 2½ weeks (days 18, 21, 25, 28 and 32).

On day 18, a master list was prepared of each mouse describing the treatment received. Mice were scored in a blinded fashion by two investigators 3× per week. Mice were followed out to day 36 at which time the study was unblinded.

The Mann Whitney U non-parametric test was used to identify significant differences between treatment groups. Differences were considered statistically significant when the P value was <0.05.

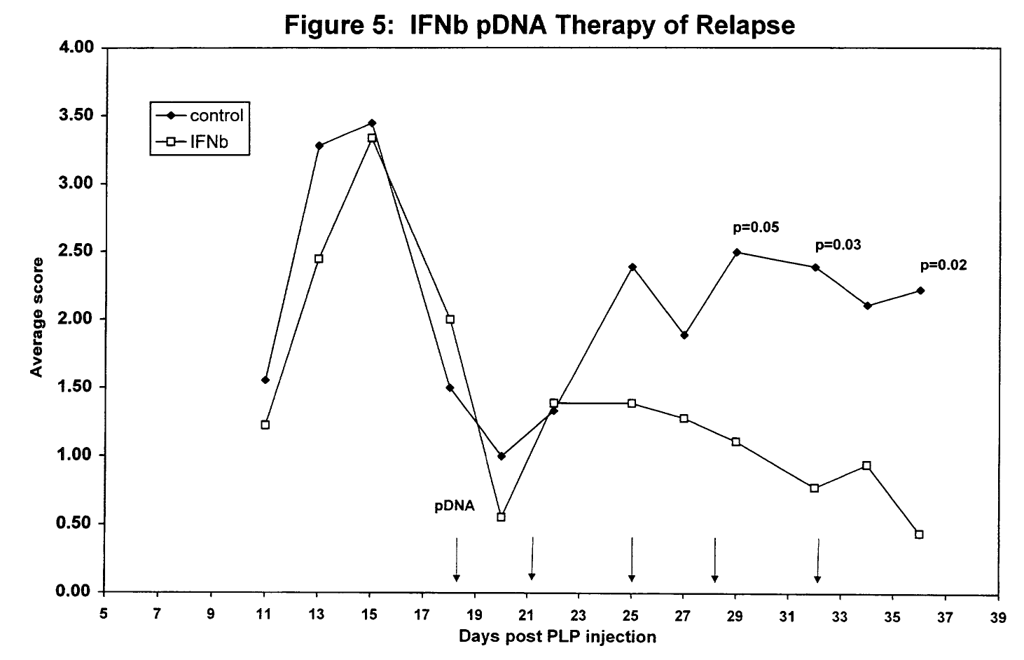

Mice receiving 100 mg IFNβ-encoding pDNA twice per week for 2½ weeks after the primary attack had a significant reduction in neurological symptoms during the relapse phase of the disease (p<0.05) (FIG. 5). The results of these studies demonstrate that IFNβ-encoding pDNA therapy can be effective in mice with established disease. The IFNβ-encoding pDNA therapy initiated after mice experienced the primary attack was able to significantly reduce neurological symptoms in the relapse phase of the disease.

### Example 8

### Intramuscular IFNβ pDNA Therapy of Experimental Autoimmune Neuritis (EAN) in Lewis Rats

The complete coding region of the rat beta-interferon gene (nucleotide 1 to 959 of GenBank accession number D87919 (SEQ ID NO:21), having the translated amino acid sequence (SEQ ID NO:22), is PCR amplified from human genomic DNA using appropriate primers.

SEQ ID NO: 21

```
    1 tggtaattaa tgaaactgca taaagttttt ataaatctct acagtttgca tacattttaa
  61 tccagtgaat agtatataaa atagccagga gcttaaataa aatgaatatt agaagctgtt
121 agaataagag aaaaatgacg gaggaaaact gaaagggaga actgaaagtg ggaaattcct
181 ctgaggcaga aaggaccatc ccttataaat agcacagacc atgaaggaag atcattctca
241 ctgcagcctt tggtagcctt tgcctcatcg tgcaggtagc agccaacacc agcccagctt
301 ccatcatggc caacaggtgg accctccaca ttgcgttcct gctgtgcttc tccaccactg
361 ccctctccat cgactacaag cagctccagt tccgacaaag cactagcatt cggacatgtc
421 agaagctcct gaggcagctg aatggaaggc tcaacctcag ctacaggacg gacttcaaga
481 tccctatgga ggtgatgcac ccgtcacaga tggagaagag ttacactgcc tttgccattc
541 aagtgatgct ccagaatgtc tttcttgtct tcagaagcaa tttctccagc actgggtgga
601 atgagactat tgttgaaagt ctcttggatg aactacatca gcagacagag cttctggaga
661 taatactaaa ggaaaagcaa gaggaaagat tgacttgggt gacatccacg actactttag
721 gcttgaagag ctattactgg agggtacaaa ggtaccttaa agacaagaag tacaacagct
781 atgcctggat ggtggtccga gcagaagtct tcaggaactt ttccattatt ctaagactta
841 atagaaactt ccagaactga agacctgtca gccaatgcct ccaagagcag gtgatggttg
901 caggcaatct taaaacatta gagtctgact ctgtgactgg tagtgaatct actgcattt
```

SEQ ID NO: 22

MANRWTLHIAFLLCFSTTALSIDYKQLQFRQSTSIRTCQKLLRQLNGRLN
LSYRTDFKIPMEVMHPSQMEKSYTAFAIQVMLQNVFLVFRSNFSSTGWNE
TIVESLLDELHQQTELLEIILKEKQEERLTWVTSTTTLGLKSYYWRVQRY
LKDKKYNSYAWMVVRAEVFRNFSIILRLNRNFQN

The PCR product is digested with EcoRI plus NheI and ligated into the same sites of the expression vector VR1051. Alternative vectors, e.g VR1055, may also be used. VR1051 and VR1055 contain a kanamycin-resistance gene, a human cytomegalovirus (CMV) immediate early gene I promoter/enhancer, 5′ untranslated sequence, CMV intron A sequence and a transcriptional terminator region derived from the rabbit β-globin gene (Hartikka, J., et al., Hum. Gene Ther. 7:1205-1217 (1996)). Verification of biological activity of the rat IFNβ-encoding plasmid is tested and pDNA is prepared by bacterial fermentation and purified as described in Example 5.

EAN, a well-known animal model of human Guillain-Barre syndrome (GBS) (see, e.g., Zou, L. P., et al, J. Neurosci. Res. 56(2):123-30 (1999)), is induced in 6 week old male Lewis rats on day 0. On day 0, rats are injected in the right hind footpad with 100 μg of a purified neurotropic epitope derived from peripheral nerve myelin protein-22 (PMP22) (for example, amino acids 53-64, Cys-Phe-Ser-Ser-Ser-Pro-Asn-Glu-Trp-Leu-Gln-Ser) (SEQ ID NO: 14) and Freund's incomplete adjuvant with 500 μg Mycobacterium tuberculosis. Alternatively, neurotropic epitopes derived from myelin basic protein-2 (P2) (for example, amino acids 58-73) (Kadlubowski, M., et al., Nature 277:140-141 (1979); Rostami, A. M., et al., Neurology 38 Suppl 1:375 (1988); Olee, T., et al, J. Neuroimmunol 21:235-240 (1989); Hahn, A. F., et al., Acta Neuropathol (Berl) 82:60-65 (1991)) or myelin glycoprotein P0 (Linington C. et al., J. Immunol 137:3826-3831 (1986), may be administered intravenously or as described above. The peptide/M. tuberculosis emulsion is made as follows: 100 μg peptide is diluted in 50 μl saline, 500 μg M. tuberculosis diluted in 50 μl of Freund's Incomplete Adjuvant. The 2 solutions are mixed with tissue homogenizer until a stable emulsion is formed. After anesthetizing the rats with metophane, each rat is injected in the right hind footpad with 100 μl of the emulsion on day 0 with a 1 cc syringe fitted with a 22 g needle. On day 0, 2 treatment groups are created having equal weights per group and a master list of treatment/rats is created.

On day 0 and 10, rats are given a bilateral im injection in the rectus femoris of 100 μg VR1051 or V1055 (control) or VR4121 (mIFNβ) in 100 μl of 150 mM sodium phosphate, pH 7.2 to which 0.01% Pluronic®-R 25R2 was added immediately prior to injection, as described in Example 6. On day 15, rats are randomized to allow for scoring in a blind fashion and the rats are scored daily for neurological symptoms, including tail tip weakness, and weight according to an 18-point protocol as described in (Gabriel, C. M. et al., Brain 120:1533-1540 (1997)), which is herein incorporated by reference in its entirety. On day 15, the study is unblinded. A terminal electromyogram (EMG) examination is performed on the animals in each group. Specifically, the left sciatic and tibial nerves are stimulated with supramaximal electrical stimuli delivered at the sciatic notch and ankle using monopolar needle electrodes (stimulus duration 0.1 ms, 2× supramaximal voltage, 1 Hz). Recordings are made of the EMG signal from the dorsum of the left hind foot. The magnitude and latency of the compound motor action potentials (CMAPs) obtained from proximal and distal stimulation are determined, and motor nerve conduction velocity is calculated. Following EMG recording, blood samples (2-5 ml) were taken from each animal by cardiac puncture and serum was analyzed by ELISA for antibodies against the immunizing antigen used. The cauda equina and left sciatic nerve are removed, processed into resin, stained with thionin acetate and acridine orange and are graded upon histological examination for oedema, axonal degeneration, demyelination, and cellular infiltration on the following scale:

TABLE 3
The Histological Analysis EAN Scoring Guide

| Score | Signs |
|---|---|
| 0 | normal |
| 1 | mild, with, 10% of the cross-sectional area/1-9 myelinated |
| | nerve fibres affected |
| 2 | moderate, with 10-50% of the cross-sectional area/10-50 |
| | myelinated fibres affected |
| 3 | severe, with >50% of the cross-sectional area/>50 myelinated nerve |
| | fibres affected |

### Example 9

### Intramuscular IFNβ pDNA Therapy of Relapsing EAN

EAN is induced in Lewis rats on day 0 as described in Example 8. The rats are monitored daily by determining neurological score and weight. On day 13, rats are selected for each treatment group based on neurological scores in the primary attack. Each treatment group contains rats with an equivalent range of scores such that graphs of average score per group overlap. Weights are checked and treatment groups contain rats of equivalent weights. A master list of treatment/rat is created. On days 13, 15, 17 and 19, rats are given a bilateral i.m. injection in the rectus femoris of 100 μg VR1051 or V1055 (control) or 100 μg VR4121 (IFNβ) in 150 mM sodium phosphate in 100 μl of 150 mM sodium phosphate, pH 7.2 to which 0.01% Pluronic®-R 25R2 is added immediately prior to injection, as described in Example 6. Rats continue to be scored and weighed, as described in Example 8, during this time period 3 times per week. On day 40, the study is unblinded. A terminal EMG examination and histological analysis is performed as described in Example 8. Histological analysis is carried out according to the EAN scoring guide, shown in Table 3.

### Example 10

### IFNβ pDNA Therapy for Guillain-Barre Syndrome in Humans

Guillain-Barre syndrome (GBS), is manifested as autoimmune inflammation of the peripheral nervous system in human patients. To treat GBS (e.g., primary progressive, secondary progressive, or relapsing-remitting), 10 ng-30 mg, preferably 200 μg-20 mg, preferably 1-10 mg of IFNβ-encoding plasmid DNA (e.g., VR6237) in a pharmacologically acceptable carrier is delivered to patients one to three times during the first week of treatment and intermittently thereafter (e.g., biweekly or monthly) by injection, preferably intramuscularly. The therapy regimen is continued indefinitely, or at least for three months to one year, during which time the patients are monitored for (a) Kurtzke Expanded Disability Status Scale (EDSS) score changes from baseline (Kurtzke, J. F., Neurology 33:1444-1452 (1983); (b) annual exacerbation rates; and (c) the severity of exacerbation based in the Scripps Neurologic Rating Scale (NRS; Sipe, J. C., et al., Neurology 34:1368-1372 (1984).

A current treatment of GBS is plasma exchange (plasmapheresis) which temporarily removes inflammatory mediators, autoantibodies, and restores normal proportions of T cell subsets. Another GBS treatment is intravenous immunoglobulin, however there have been reports of renal failure. Injection of IFNβ pDNA may avoid these temporary, involved, costly treatments and may not require renal function monitoring during treatment.

### Example 11

### Intramuscular IFNβ pDNA Therapy of Collagen-Type II Induced Arthritis (CIA) in Rhesus Monkeys (Macaca mulatta)

CIA, a well-known animal model of human rheumatoid arthritis (RA), is induced in rhesus monkeys that are susceptible to CIA based on the lack of the MHC class I allele Mamu-A26 (see, e.g., Tak, P., e al., Rheumatology 38:362-369 (1999)). On day 0, monkeys are injected intracutaneously on the back with 1 mL of 3 mg/ml of type II collagen from bovine hyaline cartilage (B-CII) and complete Freund's adjuvant. Specifically, B-CII is dissolved in 0.1 M acetic acid into a clear solution with a final concentration of 6 mg/mil of BC-II and then emulsified in an equal volume of complete Freund's adjuvant. Each monkey is injected in intracutaneously on the back with 1 ml of the emulsion, which is distributed over 10 spots (0.1 ml per spot). On day 0, 2 treatment groups are created having equal weights per group and a master list of treatment/monkey is created.

On day 0 and 30, monkeys are injected, preferably intramuscularly, with 10 ng-30 mg, preferably 200 μg-20 mg, preferably 1-10 mg of IFNβ-encoding plasmid DNA (e.g., VR6237) or control DNA (e.g. VR1055 or VR1051) in 100 μl of 150 mM sodium phosphate, pH 7.2 to which 0.01% Pluronic®-R 25R2 is added immediately prior to injection, as described in Example 6. On day 30, monkeys are randomized to allow for scoring in a blind fashion and the monkeys are scored daily for apparent symptoms of arthritis, including soft-tissue swelling (STS) and redness of affected joints, based on the following scale:

TABLE 4
The CIA Scoring Guide

| Score | Signs |
|---|---|
| 0 | no disease symptoms |
| 0.5 | fever |
| 1 | apathy and loss of appetite, weight loss |
| 2 | weight loss, warm joints with pain, no apparent joint swelling |
| 3 | moderate STS, but normal flexibility of affected joints |
| 4 | severe STS with joint stiffness |
| 5 | severe disease necessitating euthanasia |

### Example 9

### Intramuscular IFNβ pDNA Therapy of Relapsing CIA

CIA is induced in mice on day 0 as described in Example 8. The monkeys are monitored daily by determining neurological score and weight. On day 30, mice are selected for each treatment group based on CIA scores in the primary attack. Each treatment group contains monkeys with an equivalent range of scores such that graphs of average score per group overlap. Weights are checked and treatment groups contain mice of equivalent weights. A master list of treatment/monkey is created. On day 30, 33, 40 and 47 monkeys are injected, preferably intramuscularly, with 10 ng-30 mg, preferably 200 μg-20 mg, preferably 1-10 mg of IFNβ-encoding plasmid DNA (e.g., VR6237) or control DNA (e.g VR1055 or VR1051) in 100 μl of 150 mM sodium phosphate, pH 7.2 to which 0.01% Pluronic®-R 25R2 was added immediately prior to injection, as described in Example 6. Monkeys continue to be scored and weighed, as described in Example 8, during this time period 3 times per week. On day 50, the study is unblinded.

### Example 10

### IFNβ pDNA Therapy for Active Rheumatoid Arthritis in Humans

Rheumatoid Arthritis (RA), is manifested as autoimmune inflammationin the joints of human patients. To treat RA (e.g., primary progressive, secondary progressive, or relapsing-remitting), ^{˜}10 μg-30 mg, preferably 200 μg-20 mg, preferably 1-10 mg of IFNβ plasmid DNA (e.g., VR6237) in a pharmacologically acceptable carrier is delivered to patients one to three times during the first week of treatment and intermittently thereafter (e.g., biweekly or monthly) by injection, preferably intramuscularly. The therapy regimen is continued indefinitely, or at least for three months to one year. The patients are assessed for response to IFNβ plasmid DNA at day 5, 8, 15, 22, 29, 43, 57, 71 and 85, during which time the patients are monitored for vital signs, duration of morning stiffness (minutes), tender joint count (68 joints), swollen joint count (66 joints), patient's assessment of pain [0-10 cm on a visual analogue scale (VAS)], patient's global assessment (VAS 0-10 cm), physician's global assessment (VAS 0-10 cm) and the Health Assessment Qustionnaire (HAQ). In addition, standard laboratory tests are performed, including ESR, serum levels of CRP, a complete blood cell count, serum electrolytes, glucose, creatinine, urea, alkaline phosphatase, aspartame transaminase, total bilirubin, lactate dehydrogenate and serum albumin. Urine analysis is also performed at each assessment point. Before study entry and at day 85, the following investigations are performed: serum levels of rheumatoid factor by ELISA, antinuclear antibodies on Hep-2 cells and antibodies to double-stranded DNA on critidiae by immunofluorescence.

It is clear that the invention may be practiced otherwise than as particulary described in the foregoing description and examples.

Numerous modifications and variations of the present invention are possible in light of the above teachings and, therefore, are within the scope of the appended claims.

The entire disclosure of all publications (including patents, patent application, journal articles, laboratory manuals, books, or other documents) cited herein are hereby incorporated by reference in their entireties.

It is clear that the invention may be practiced otherwise than as particular described in the foregoing description and examples.

What is claimed is:

1. A method of treating an inflammatory demyelinating disease in a mammal comprising:
administering in vivo into a tissue of a mammal a composition comprising a polynucleotide encoding interferon-beta (IFNβ), or an active fragment or variant thereof; and a pharmaceutically acceptable carrier;
wherein said polynucleotide is selected from the group consisting of a DNA plasmid encoding said IFNβ or active fragment or variant thereof through operable association with a promoter, and a messenger RNA;
wherein said polynucleotide is free from association with liposomal formulations and charged lipids;
wherein said polynucleotide is incorporated into the cells of said mammal; and
wherein a therapeutically effective amount of IFNβ or active fragment or variant thereof is expressed.

2. The method of claim 1, wherein said inflammatory demyelinating disease is selected from the group consisting of multiple sclerosis, Guillain-Barre Syndrome, experimental autoimmune encephalomyelitis and experimental autoimmune neuritis.

3. The method of claim 2, wherein said inflammatory demyelinating disease is multiple sclerosis.

4. The method of claim 1, wherein said tissue is selected from the group consisting of muscle, skin, brain tissue, lung tissue, liver tissue, spleen tissue, bone marrow tissue, thymus tissue, heart tissue, lymph tissue, blood tissue, bone tissue, connective tissue, mucosal tissue, pancreas tissue, kidney tissue, gall bladder tissue, intestinal tissue, testicular tissue, ovarian tissue, uterine tissue, vaginal tissue, rectal tissue, nervous system tissue, eye tissue, glandular tissue, and tongue tissue.

5. The method of claim 4, wherein said tissue is muscle.

6. The method of claim 1, wherein said administration is by injection.

7. The method of claim 1, wherein said mammal is a human.

8. The method of claim 1, wherein said IFNβ or active fragment or variant thereof is human IFNβ or and active fragment or variant thereof.

9. The method of claim 1, wherein said polynucleotide comprises a nucleic acid selected from the group consisting of:
(a) a nucleic acid that hybridizes under stringent conditions to the complement of SEQ ID NO:1, wherein the polynucleotide sequence encodes a polypeptide that has anti-viral or anti-proliferative activity;
(b) a nucleic acid that encodes a polypeptide which, except for at least one but not more than 20 individual amino acid substitutions, deletions, or insertions, is identical to amino acids 1 to 166 in SEQ ID NO:2, wherein the polypeptide has anti-viral or anti-proliferative activity; and
(c) a nucleic acid that encodes a polypeptide at least 95% identical to amino acids 1 to 166 in SEQ ID NO:2, wherein the polypeptide has anti-viral or anti-proliferative activity.

10. The method of claim 9, wherein said polynucleotide comprises a nucleic acid which encodes amino acids 1 to 166 of SEQ ID NO:2.

11. A method of treating an inflammatory demyelinating disease in a mammal comprising:
administering in vivo into a tissue of a mammal a composition comprising a polynucleotide encoding interferon-beta (IFNβ), or an active fragment or variant thereof; and a pharmaceutically acceptable carrier;
wherein said polynucleotide is selected from the group consisting of a DNA plasmid encoding said IFNβ or active fragment or variant thereof through operable association with a promoter, and a messenger RNA;
wherein said tissue is selected from the group consisting of muscle, skin, or blood;
wherein said polynucleotide is incorporated into the cells of said mammal; and
wherein a therapeutically effective amount of IFNβ is expressed.

12. The method of claim 11, wherein said inflammatory demyelinating disease is selected from the group consisting of multiple sclerosis, Guillain-Barre Syndrome, experimental autoimmune encephalomyelitis and experimental autoimmune neuritis.

13. The method of claim 12, wherein said inflammatory demyelinating disease is multiple sclerosis.

14. The method of claim 11, wherein said tissue is muscle.

15. The method of claim 11, wherein said tissue is skin.

16. The method of claim 11, wherein said tissue is blood.

17. The method of claim 11, wherein said administration is by injection.

18. The method of claim 11, wherein said mammal is a human.

19. The method of claim 11, wherein said IFNβ or active fragment or variant thereof is human IFNβ or and active fragment or variant thereof.

20. The method of claim 11, wherein said polynucleotide comprises a nucleic acid selected from the group consisting of.
(a) a nucleic acid that hybridizes under stringent conditions to the complement of SEQ ID NO:1, wherein the polynucleotide sequence encodes a polypeptide that has anti-viral or anti-proliferative activity;
(b) a nucleic acid that encodes a polypeptide which, except for at least one but not more than 20 individual amino acid substitutions, deletions, or insertions, is identical to amino acids 1 to 166 in SEQ ID NO:2, wherein the polypeptide has anti-viral or anti-proliferative activity; and
(c) a nucleic acid that encodes a polypeptide at least 95% identical to amino acids 1 to 166 in SEQ ID NO:2, wherein the polypeptide has anti-viral or anti-proliferative activity.

21. The method of claim 20, wherein said polynucleotide comprises a nucleic acid which encodes amino acids 1 to 166 of SEQ ID NO:2.
